(19) United States
(12) Patent Application Publication
(10) Pub. No.: US 2008/0274947 A1
(43) Pub. Date: Nov. 6, 2008

(54) Hydroxy-Substituted Diphenylazetidinones for the Treatment of Hyperlipidemia

(75) Inventors: Gerhard JAEHNE, Frankfurt am Main (DE); Wendelin FRICK, Frankfurt am Main (DE); Andreas LINDENSCHMIDT, Frankfurt am Main (DE); Hubert HEUER, Frankfurt am Main (DE); Hans-Ludwig SCHAEFER, Frankfurt am Main (DE); Werner KRAMER, Frankfurt am Main (DE); Wolfgang SCHMIDER, Frankfurt am Main (DE)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS DEUTSCHLAND GMBH, Frankfurt am Main (DE)

(21) Appl. No.: 12/122,991

(22) Filed: May 19, 2008

Related U.S. Application Data
(63) Continuation of application No. PCT/EP2006/010840, filed on Nov. 13, 2006.

(30) Foreign Application Priority Data
Nov. 23, 2005 (DE) 102005055726.0

(51) Int. Cl. A61K 38/28; C07D 403/12; A61P 3/10; A61P 9/10; A61K 31/496

(52) U.S. Cl. 514/4; 540/200; 514/210.02

(57) ABSTRACT

The present invention comprises compounds and compositions for the treatment of metabolic disorders and more particularly, those insulin-related metabolic disorders of the blood such as hyperlipidemia, diabetes, insulin-resistance and the like comprising diphenylazetidinones compounds which have an additional hydroxy function in the 2″ position and their salts. The invention therefore relates to compounds of formula I:

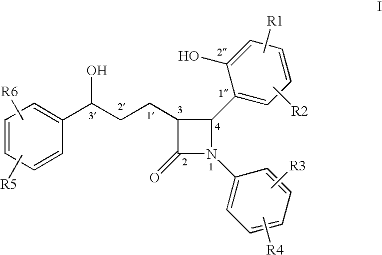

in which the meanings R1, R2, R3, R4, R5, R6 are defined herein.

### CROSS-REFERENCE TO RELATED APPLICATIONS

This application is a continuation application of International Patent Application No. PCT/EP2006/010840 filed on Nov. 13, 2006 which is incorporated herein by reference in its entirety which also claims the benefit of priority of German patent application Ser. No. 10/2005 055726.0 filed on Nov. 23, 2005.

### FIELD OF THE INVENTION

The present invention relates generally to pharmaceutical compositions for the treatment of metabolic disorders and in particular hyperlipidemia and related cardiovascular disorders of the blood. The invention relates generally to compounds and compositions for the treatment of metabolic disorders and more particularly, those insulin-related metabolic disorders of the blood such as hyperlipidemia and diabetes and the like. More specifically, the present invention relates to compounds that therapeutically modulation and control lipid and/or carbohydrate metabolism and are thus suitable for the prevention and/or treatment of diseases such as type-2 diabetes, atherosclerosis, and the diverse manifestations thereof. More specifically, the present invention relates to diphenylazetidinones which are substituted by hydroxyl functions and to the pharmaceutically acceptable salts thereof.

### BACKGROUND OF THE INVENTION

The compounds of the present invention are highly effective in the therapeutic modulation of lipid and/or carbohydrate metabolism and are therefore useful in the prevention and/or treatment of diseases such as type-2 diabetes and atherosclerosis, and the many other diverse cardiovascular manifestations therefrom. More specifically, the present invention relates to diphenylazetidinones which are substituted by hydroxyl functions and pharmaceutically acceptable salts thereof. Diphenylazetidinones, and their use in the treatment of hyperlipidemia have been described in the prior art, (see PCT/EP03/05816, PCT/EP03/05815 and PCT/EP03/05816). These compounds however, are not easily administered and their utility is limited by their low rate of absorption. The compounds of the present invention however, exhibit a therapeutically utilizable hypolipidemic effect, and, in particular, show lower liver levels compared with those compounds described in the prior art. The lower liver levels reduce the possibility of liver stress by the active and thereby reduce the likelihood of drug-drug interaction. This objective is achieved by diphenylazetidinones compounds which have an additional hydroxy function in the 2″ position.

### SUMMARY OF THE INVENTION

The present invention comprises compounds and compositions for the treatment of metabolic disorders and more particularly, those insulin-related metabolic disorders of the blood such as hyperlipidemia, diabetes, insulin-resistance and the like comprising diphenylazetidinones compounds which have an additional hydroxy function in the 2″ position and their salts. The invention therefore relates to compounds of formula I:

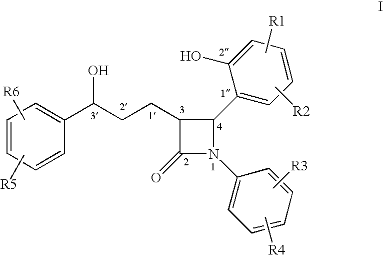

in which the meanings of R1, R2, R3, R4, R5, and R6 are defined herein.

### DETAILED DESCRIPTION OF THE INVENTION

The present invention comprises compounds and compositions for the treatment of metabolic disorders and more particularly, those insulin-related metabolic disorders of the blood such as hyperlipidemia, diabetes, insulin-resistance and the like comprising diphenylazetidinones compounds which have an additional hydroxy function in the 2″ position and their salts. The invention therefore relates to compounds of formula I:

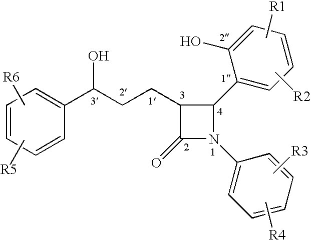

wherein:
R1, R2, R3, R4, R5 and R6 are independently of one another selected from the group consisting of (C₁-C₃₀)-alkylene-(LAG)ₙ, where n may be 1-5, and where one or more C atoms of the alkylene group may be replaced by —S(O)ₙ—, with n=0-2, —O—, —(C═O)—, —(C═S)—, —CH═CH—, —C≡C—, —N((C₁-C₆)-alkyl)-, —N(phenyl)-, —N((C₁-C₆)-alkylphenyl)-, —N(CO—(CH₂)₁₋₁₀—COOH)—, —N(CO—(C₁-C₈)-alkyl)-, —N(CO—(C₃-C₈)-cycloalkyl), N(CO—(CH₂)₀₋₁₀-aryl), —N(CO—(CH₂)₀₋₁₀-heteroaryl), —NH— or by aryl or heteroaryl groups which may be substituted up to three times by R7, or by (C₃-C₁₀)-cycloalkyl or heterocycloalkyl groups which may be substituted up to four times by R7; H, F, Cl, Br, I, CF₃, NO₂, N₃, CN, COOH, COO(C₁-C₆)alkyl, CONH₂, CONH(C₁-C₆)alkyl, CON[(C₁-C₆)alkyl]₂, (C₁-C₆)-alkyl, (C₂-C₆)-alkenyl, (C₂-C₆)-alkynyl, O—(C₁-C₆)-alkyl, where one, more than one, or all hydrogen(s) in the alkyl groups may be replaced by fluorine; CH(═NH)(NH₂), PO₃H₂, SO₃H, SO₂—NH₂, SO₂NH(C₁-C₆)-alkyl, SO₂N[(C₁-C₆)-alkyl]₂, S—(C₁-C₆)-alkyl, S—(CH₂)ₙ-phenyl, SO—(C₁-C₆)-alkyl, SO—(CH₂)ₙ-phenyl, SO₂—(C₁-C₆)-alkyl, SO₂—(CH₂)ₙ-phenyl, where n may be 0-6, and the phenyl group may be substituted up to twice by F, Cl, Br, OH, CF₃, NO₂, CN, OCF₃, O—(C₁-C₆)-alkyl, (C₁-C₆)-alkyl, NH₂;
NH₂, NH—(C₁-C₆)-alkyl, N((C₁-C₆)-alkyl)₂, NH(C₁-C₇)-acyl, phenyl, O—(CH₂)ₙ-phenyl, where n may be 0-6, where the phenyl ring may be substituted one to three times by F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl, CONH₂;
R7 is selected from the group consisting of F, Cl, Br, I, CF₃, NO₂, N₃, CN, COOH, COO(C₁-C₆)alkyl, CONH₂, CONH(C₁-C₆)alkyl, CON[(C₁-C₆)alkyl]₂, (C₁-C₆)-alkyl, (C₂-C₆)-alkenyl, (C₂-C₆)-alkynyl, O—(C₁-C₆)-alkyl, where one, more than one, or all hydrogen(s) in the alkyl groups may be replaced by fluorine; C(═NH)(NH₂), PO₃H₂, SO₃H, SO₂—NH₂, SO₂NH(C₁-C₆)-alkyl, SO₂N[(C₁-C₆)-alkyl]₂, S—(C₁-C₆)-alkyl, S—(CH₂)ₙ-phenyl, SO—(C₁-C₆)-alkyl, SO—(CH₂)ₙ-phenyl, SO₂—(C₁-C₆)-alkyl, SO₂—(CH₂)ₙ-phenyl, where n may be 0-6, and the phenyl group may be substituted up to twice by F, Cl, Br, OH, CF₃, NO₂, CN, OCF₃, O—(C₁-C₆)-alkyl, (C₁-C₆)-alkyl, NH₂;
   NH₂, NH—(C₁-C₆)-alkyl, N((C₁-C₆)-alkyl)₂, NH(C₁-C₇)-acyl, aryl, O—(CH₂)ₙ-aryl, where n may be 0-6, where the aryl ring may be substituted one to three times by F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl, CONH₂;

where at least one of the substituents R1 to R6 must always have the meaning of (C₁-C₃₀)-alkylene-(LAG)ₙ, where n may be 1-5, and where one or more C atoms of the alkylene group may be replaced by —S(O)ₙ—, with n=0-2, —O—, —(C═O)—, —(C═S)—, —CH═CH—, —C≡C—, —N((C₁-C₆)-alkyl)-, —N(phenyl)-, —N((C₁-C₆)-alkylphenyl)-, —N(CO—(CH₂)₁₋₁₀—COOH)—, —N(CO—(C₁-C₈)-alkyl)-, —N(CO—(C₃-C₈)-cycloalkyl), N(CO—(CH₂)₀₋₁₀-aryl), —N(CO—(CH₂)₀₋₁₀-heteroaryl), —NH— or by aryl or heteroaryl groups which are substituted up to three times by R7, or by (C₃-C₁₀)-cycloalkyl or heterocycloalkyl groups which are substituted up to four times by R7;

The abbreviation “LAG” above and herein throughout designates a C₄-C₁₀-cyclo-aliphatic group substituted by 2 to 9 hydroxyl (—OH) functions, or C₂-C₁₀-aliphatic groups substituted with 2 to 10 hydroxyl functions, where in each case one or more hydroxyl functions may be replaced by an —NHR8 group; an amino acid residue, oligopeptide residues consisting of 2 to 9 amino acids; acyclic, mono- or bicyclic trialkyl-ammonium groups, acyclic, mono- or bicyclic trialkyl-ammoniumalkyl groups, where up to three carbon atoms may be replaced by N, O or S(O), with n=0-2; N-alkylated heteroaromatics such as, for example, imidazolium or pyridinium; O—(SO₂)—OH; —(CH₂)₀₋₁₀—SO₃H; —(CH₂)₀₋₁₀—P(O)(OH)₂, —(CH₂)₀₋₁₀—P(O)(OH)₂, —(CH₂)₀₋₁₀—C(═NH)(NH₂); —(CH₂)₀₋₁₀—C(═NH)(NHOH); —NR8-C(═NR9)(NR10R11); where n is 1-5, and R8, R9, R10 and R11 may independently of one another be H, (C₁-C₆)-alkyl, phenyl, (C₁-C₆)-alkyl-phenyl, (C₃-C₈)-cycloalkyl, —C(O)—(C₁-C₆)-alkyl, —C(O)—(C₃-C₈)-cycloalkyl; and the pharmaceutically acceptable salts thereof.

Preferred compounds of formula I of the present invention are those in which at least one of the side groups R1 to R6 has the meaning of (C₁-C₂₀)-alkylene-(LAG), where one or more C atoms of the alkylene group may be replaced by —O—, —(C═O)—, —N((C₁-C₆)-alkyl)-, —N(CO—(CH₂)₁₋₁₀—COOH)— or —NH— or by aryl or heteroaryl groups which are substituted up to three times by R7, or by (C₃-C₁₀)-cycloalkyl or heterocycloalkyl groups which are substituted up to four times by R7.

More preferred compounds of the formula I are those in which one of the groups R1 or R3 has the meaning of (C₁-C₁₂)-alkylene-(LAG), where one or more C atoms of the alkylene group may be replaced by —O—, —(C═O)—, —N(CH₃)—, or —NH— or by aryl or heteroaryl groups which are substituted up to three times by R7, or by (C₃-C₁₀)-cycloalkyl or heterocycloalkyl groups which are substituted up to four times by R7.

Very preferred compounds of the present invention of formula I are those in which one of the R1 or R3 groups has the meaning of (C₁-C₅)-alkylene-(LAG); in which one or more carbon atoms of the alkylene moiety group may be replaced by —O—, —(C═O)— or —NH— or by aryl or heteroaryl groups which are substituted up to three times by R7, or by (C₃-C₁₀)-cycloalkyl or heterocycloalkyl groups which are substituted up to four times by R7.

Even more preferred compounds of formula I of the present invention are those in which one of the groups R1 or R3 designates —O—CH₂-aryl-CH₂-(LAG), —CH₂—O—(C═O)-heterocycloalkyl-(LAG), —CH₂—NH—(C═O)-heterocycloalkyl-(C═O)—CH₂-(LAG), —CH₂-heterocycloalkyl-(LAG), —NH—(C═O)-heterocycloalkyl-(LAG) or —O—(C═O)-heterocycloalkyl-(LAG).

Another group of preferred compounds of formula I are those in which one of the groups R1 or R3 has the meaning of —CH₂—O—(C═O)-heterocycloalkyl-(LAG), —CH₂—NH—(C═O)-heterocycloalkyl-(C═O)—CH₂-(LAG), —CH₂-heterocycloalkyl-(LAG), —NH—(C═O)-heterocycloalkyl-(LAG) or —O—(C═O)-heterocycloalkyl-(LAG).

Additional preferred compounds of the formula I are those in which one of the groups R1 or R3 has as heterocycloalkyl group the 1,4-piperazinediyl group

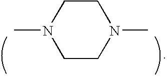

Most preferred compounds of the formula I are those in which the group LAG is a sulfate residue (—O—SO₃H), a sulfonic acid residue (—SO₃H), a mono- or bicyclic cycloalkyl group in which one or more carbons may be replaced by nitrogen, or a mono- or bicyclic tri-alkyl-ammonium-alkyl group.

A mono- or bicyclic trialkyl-ammonium group means a mono- or bicyclic cycloalkyl group in which one or more carbons are replaced by nitrogen, and the nitrogen carries an additional hydrogen and positive charge.

For example groups such as

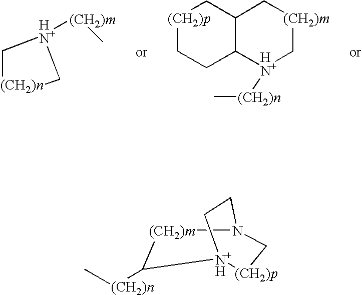

where n, m and p are independently of one another 0-10, and one or more CH₂ groups may be replaced independently of one another by O, S(O)ₙ, where n may be 0-2, NH, N—(C₁-C₁₀)-alkyl, N-phenyl or N—CH₂-phenyl.

A mono- or bicyclic trialkyl-ammoniumalkyl group means a mono- or bicyclic cycloalkyl group in which one or more carbons are replaced by nitrogen, and the nitrogen carries an additional alkyl group and positive charge.

For example groups such as

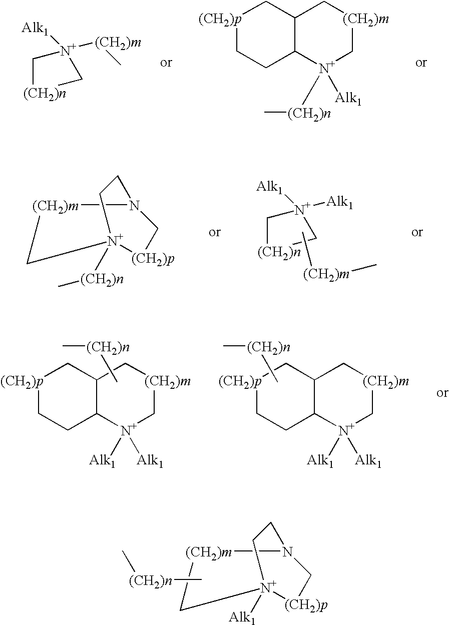

where n, m and p may independently of one another be 0-10, and one or more CH₂ groups may independently of one another be replaced by O, S(O)ₙ, where n may be 0-2, NH, N—(C₁-C₁₀)-alkyl, N-phenyl or N—CH₂-phenyl, and Alk₁ is a linear or branched alkyl group having 1 to 20 carbon atoms.

N-Alkylated heteroaromatics mean groups such as, for example,

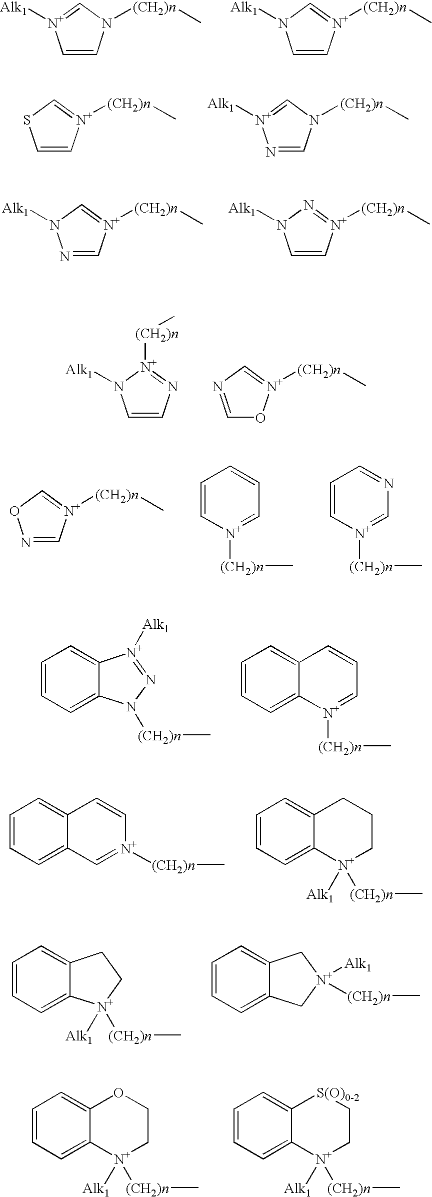

where n may be 0-10, and Alk₁ is a linear or branched alkyl group having 1 to 20 carbon atoms.

Pharmaceutically acceptable salts are, because their solubility in water is greater than that of the initial or basic compounds, particularly suitable for medical applications.

These salts must have a pharmaceutically acceptable anion or cation. Suitable pharmaceutically acceptable acid addition salts of the compounds of the invention are salts of inorganic acids such as hydrochloric acid, hydrobromic acid, phosphoric, metaphosphoric, nitric, sulfonic and sulfuric acids, and of organic acids such as, for example, acetic acid, benzenesulfonic, benzoic, citric, ethanesulfonic, fumaric, gluconic, glycolic, isethionic, lactic, lactobionic, maleic, malic, methane-sulfonic, succinic, p-toluenesulfonic, and tartaric acids. The chlorine salt is particularly preferably used for medical purposes.

Suitable pharmaceutically acceptable basic salts are ammonium salts, alkali metal salts (such as sodium and potassium salts), alkaline earth metal salts (such as magnesium and calcium salts), zinc salts, and salts of trometamol (2-amino-2-hydroxymethyl-1,3-propanediol), diethanolamine, lysine, arginine, choline, meglumine or ethylenediamine.

Salts with a pharmaceutically unacceptable anion or cation likewise belong in the context of the invention as useful intermediates for preparing or purifying pharmaceutically acceptable salts and/or for use in non-therapeutic, for example in vitro applications.

Further aspects of this invention are prodrugs of the compounds of the invention. Such prodrugs can be metabolized in vivo to a compound of the invention. These prodrugs may themselves be active or not.

The compounds of the invention may also exist in various polymorphous forms, e.g. as amorphous and crystalline polymorphous forms. All polymorphous forms of the compound according to the invention belong within the framework of the invention and are a further aspect of the invention.

All references to “compound(s) of formula I” hereinafter refer to compound(s) of the formula I as described above, and the salts, solvates and physiologically functional derivatives thereof as described herein.

The compounds of the formula I and their pharmaceutically acceptable salts and physiologically functional derivatives represent an ideal pharmaceutical for the treatment of lipid metabolism disorders, especially hyperlipidemia. The compounds of the formula I are likewise suitable for influencing the serum cholesterol level and for the prevention and treatment of arteriosclerotic manifestations.

The compound(s) of the formula (I) can also be administered in combination with further active ingredients.

The amount of a compound of formula (I) necessary to achieve the desired biological effect depends on a number of factors, for example the specific compound chosen, the intended use, the mode of administration and the clinical condition of the patient. The daily dose is generally in the range from 0.1 mg to 100 mg (typically from 0.1 mg and 50 mg) per day and per kilogram of body weight, for example 0.1-10 mg/kg/day. Tablets or capsules may contain, for example, from 0.01 to 100 mg, typically from 0.02 to 50 mg. In the case of pharmaceutically acceptable salts, the aforementioned weight data are based on the weight of the diphenylazetidinone ion derived from the salt. For the prophylaxis or therapy of the abovementioned conditions, the compounds of formula (I) may be used as the compound itself, but it is preferably in the form of a pharmaceutical composition with an acceptable carrier. The carrier must, of course, be acceptable in the sense that it is compatible with the other ingredients of the composition and is not harmful for the patient's health. The carrier may be a solid or a liquid or both and is preferably formulated with the compound as a single dose, for example as a tablet, which may contain from 0.05% to 95% by weight of the active ingredient. Other pharmaceutically active substances may likewise be present, including further compounds of formula (I). The pharmaceutical compositions of the invention can be produced by one of the known pharmaceutical methods, which essentially consist of mixing the ingredients with pharmacologically acceptable carriers and/or excipients.

Pharmaceutical compositions of the invention are those suitable for oral and peroral (for example sublingual) administration, although the most suitable mode of administration depends in each individual case on the nature and severity of the condition to be treated and on the nature of the compound of formula (I) used in each case. Coated formulations and coated slow-release formulations also belong within the framework of the invention. Preference is given to acid- and gastric juice-resistant formulations. Suitable coatings resistant to gastric juice comprise cellulose acetate phthalate, polyvinyl acetate phthalate, hydroxy-propylmethylcellulose phthalate and anionic polymers of methacrylic acid and methyl methacrylate.

Suitable pharmaceutical compounds for oral administration may be in the form of separate units such as, for example, capsules, cachets, slow-dissolving tablets or tablets, each of which contains a defined amount of the compound of formula (I); as powders or granules; as solution or suspension in an aqueous or non-aqueous liquid; or as an oil-in-water or water-in-oil emulsion. These compositions may, as already mentioned, be prepared by any suitable pharmaceutical method which includes a step in which the active ingredient and the carrier (which may consist of one or more additional ingredients) are brought into contact. The compositions are generally produced by uniform and homogeneous mixing of the active ingredient with a liquid and/or finely divided solid carrier, after which the product is shaped if necessary. Thus, for example, a tablet can be produced by compressing or molding a powder or granules of the compound, where appropriate with one or more additional ingredients. Compressed tablets can be produced by tableting the compound in free-flowing form such as, for example, a powder or granules, where appropriate mixed with a binder, glidant, inert diluent and/or one (or more) surface-active/dispersing agent(s) in a suitable machine. Molded tablets can be produced by molding the compound, which is in powder form and is moistened with an inert liquid diluent, in a suitable machine.

Pharmaceutical compositions which are suitable for peroral (sublingual) administration comprise slow-dissolving tablets which contain a compound of formula (I) with a flavoring, normally sucrose and gum arabic or tragacanth, and pastilles which comprise the compound in an inert base such as gelatin and glycerol or sucrose and gum arabic.

Further active ingredients suitable for combination products are:

All anti-diabetics which are mentioned in the Rote Liste 2005, chapter 12; all weight-reducing agents/appetite suppressants which are mentioned in the Rote Liste 2005, chapter 1; all lipid-lowering agents which are mentioned in the Rote Liste 2005, chapter 58. They may be combined with the compound of the invention of the formula I in particular for a synergistic improvement in the effect. The active ingredient combination can be administered either by separate administration of the active ingredients to the patient or in the form of combination products in which a plurality of active ingredients is present in a pharmaceutical preparation. Most of the active ingredients mentioned hereinafter are disclosed in the USP Dictionary of USAN and International Drug Names, US Pharmacopeia, Rockville 2001.

Anti-diabetics include insulin and insulin derivatives such as, for example, Lantus® (see www.lantus.com) or HMR 1964 or those described in WO2005005477 (Novo Nordisk), fast-acting insulins (see U.S. Pat. No. 6,221,633), inhalable insulins such as, for example, Exubera® or oral insulins such as, for example, IN-105 (Nobex) or Oral-lyn™ (Generex Biotechnology), GLP-1 derivatives such as, for example, exenatide, liraglutide or those which have been disclosed in WO98/08871 or WO2005027978 of Novo Nordisk A/S, in WO01/04156 of Zealand or in WO00/34331 of Beaufour-Ipsen, pramlintide acetate (Symlin; Amylin Pharmaceuticals), and orally effective hypoglycemic active ingredients.

The orally effective hypoglycemic active ingredients include preferably

sulfonylureas,
biguanidines,
meglitinides,
oxadiazolidinediones,
thiazolidinediones,
glucosidase inhibitors,
inhibitors of glycogen phosphorylase,
glucagon antagonists,
glucokinase activators,
inhibitors of fructose-1,6-bisphosphatase,
modulators of glucose transporter 4 (GLUT4),
inhibitors of glutamine-fructose-6-phosphate amidotransferase (GFAT),
GLP-1 agonists, potassium channel openers such as, for example, those which have been disclosed in WO 97/26265 and WO 99/03861 of Novo Nordisk A/S,
inhibitors of dipeptidylpeptidase IV (DPP-IV),
insulin sensitizers,
inhibitors of liver enzymes involved in stimulating gluconeogenesis and/or glycogenolysis,
modulators of glucose uptake, of glucose transport and of glucose reabsorption,
inhibitors of 11β-HSD1,
inhibitors of protein tyrosine phosphatase 1B (PTP1B),
modulators of the sodium-dependent glucose transporter 1 or 2 (SGLT1, SGLT2),
compounds which alter lipid metabolism such as antihyperlipidemic active ingredients and anti-lipidemic active ingredients,
compounds which reduce food intake,
compounds which increase thermogenesis,
PPAR and RXR modulators and
active ingredients which act on the ATP-dependent potassium channel of the beta cells.

In one embodiment of the invention, the compound of the formula I is administered in combination with an HMGCoA reductase inhibitor such as simvastatin, fluvastatin, pravastatin, lovastatin, atorvastatin, cerivastatin, rosuvastatin, L-659699.

In one embodiment of the invention, the compound of the formula I is administered in combination with a cholesterol absorption inhibitor such as, for example, ezetimibe, tiqueside, pamaqueside, FM-VP4 (sitostanol/campesterol ascorbyl phosphate; Forbes Medi-Tech, WO2005042692), MD-0727 (Microbia Inc., WO2005021497) or with compounds as described in WO2002066464 (Kotobuki Pharmaceutical Co. Ltd.), or WO2005062824 (Merck & Co.) or WO2005061451 and WO2005061452 (AstraZeneca AB).

In one embodiment of the invention, the compound of the formula I is administered in combination with a PPAR gamma agonist such as, for example, rosiglitazone, pioglitazone, JTT-501, GI 262570, R-483 or CS-011 (rivoglitazone).

In one embodiment of the invention, the compound of the formula I is administered in combination with a PPAR alpha agonist such as, for example, GW9578, GW-590735, K-111, LY-674, KRP-101, DRF-10945.

In one embodiment of the invention, the compound of the formula I is administered in combination with a mixed PPAR alpha/gamma agonist such as, for example, muraglitazar, tesaglitazar, naveglitazar, LY-510929, ONO-5129, E-3030, AVE 8042, AVE 8134, AVE 0847, or as described in PCT/US00/11833, PCT/US00/11490, DE10142734.4 or in J. P. Berger et al., TRENDS in Pharmacological Sciences 28(5), 244-251, 2005.

In one embodiment of the invention, the compound of the formula I is administered in combination with a PPAR delta agonist such as, for example, GW-501516. In one embodiment of the invention, the compound of the formula I is administered in combination with metaglidasen or with MBX-2044 or other partial PPAR gamma agonists/antagonists.

In one embodiment of the invention, the compound of the formula I is administered in combination with a fibrate such as, for example, fenofibrate, clofibrate or bezafibrate.

In one embodiment of the invention, the compound of the formula I is administered in combination with an MTP inhibitor such as, for example, implitapide, BMS-201038, R-103757, or those described in WO2005085226.

In one embodiment of the invention, the compound of the formula I is administered in combination with a CETP inhibitor such as, for example, torcetrapib or JTT-705.

In one embodiment of the invention, the compound of the formula I is administered in combination with a bile acid absorption inhibitor (see, for example, U.S. Pat. No. 6,245,744, U.S. Pat. No. 6,221,897 or WO00/61568), such as, for example, HMR 1741 or those as described in DE 10 2005 033099.1 and DE 10 2005 033100.9.

In one embodiment of the invention, the compound of the formula I is administered in combination with a polymeric bile acid adsorbent such as, for example, cholestyramine or colesevelam.

In one embodiment of the invention, the compound of the formula I is administered in combination with an LDL receptor inducer (see U.S. Pat. No. 6,342,512), such as, for example, HMR1171, HMR1586 or those as described in WO2005097738.

In one embodiment, the compound of the formula I is administered in combination with Omacor® (omega-3 fatty acids; highly concentrated ethyl esters of eicosapentaenoic acid and of docosahexaenoic acid).

In one embodiment of the invention, the compound of the formula I is administered in combination with an ACAT inhibitor such as, for example, avasimibe.

In one embodiment of the invention, the compound of the formula I is administered in combination with an antioxidant such as, for example, OPC-14117, probucol, tocopherol, ascorbic acid, β-carotene or selenium.

In one embodiment of the invention, the compound of the formula I is administered in combination with a vitamin such as, for example, vitamin B6 or vitamin B12.

In one embodiment of the invention, the compound of the formula I is administered in combination with a lipoprotein lipase modulator such as, for example, ibrolipim (NO-1886).

In one embodiment of the invention, the compound of the formula I is administered in combination with an ATP citrate lyase inhibitor such as, for example, SB-204990.

In one embodiment of the invention, the compound of the formula I is administered in combination with a squalene synthetase inhibitor such as, for example, BMS-188494 or as described in WO2005077907.

In one embodiment of the invention, the compound of the formula I is administered in combination with a lipoprotein (a) antagonist such as, for example, gemcabene (CI-1027).

In one embodiment of the invention, the compound of the formula I is administered in combination with an HM74A receptor agonist such as, for example, nicotinic acid.

In one embodiment of the invention, the compound of the formula I is administered in combination with a lipase inhibitor such as, for example, orlistat or cetilistat (ATL-962).

In one embodiment of the invention, the compound of the formula I is administered in combination with insulin.

In one embodiment of the invention, the compound of the formula I is administered in combination with a sulfonylurea such as, for example, tolbutamide, glibenclamide, glipizide or glimepiride.

In one embodiment, the compound of the formula I is administered in combination with a biguanide such as, for example, metformin.

In another embodiment, the compound of the formula I is administered in combination with a meglitinide such as, for example, repaglinide or nateglinide.

In one embodiment, the compound of the formula I is administered in combination with a thiazolidinedione such as, for example, troglitazone, ciglitazone, pioglitazone, rosiglitazone or the compounds disclosed in WO 97/41097 of Dr. Reddy's Research Foundation, in particular 5-[[4-[(3,4-dihydro-3-methyl-4-oxo-2-quinazolinylmethoxy]phenyl]methyl]-2,4-thiazolidinedione.

In one embodiment, the compound of the formula I is administered in combination with an α-glucosidase inhibitor such as, for example, miglitol or acarbose.

In one embodiment, the compound of the formula I is administered in combination with an active ingredient which acts on the ATP-dependent potassium channel of the beta cells, such as, for example, tolbutamide, glibenclamide, glipizide, glimepiride or repaglinide.

In one embodiment, the compound of the formula I is administered in combination with more than one of the aforementioned compounds, e.g. in combination with a sulfonylurea and metformin, a sulfonylurea and acarbose, repaglinide and metformin, insulin and a sulfonylurea, insulin and metformin, insulin and troglitazone, insulin and lovastatin, etc.

In one embodiment, the compound of the formula I is administered in combination with an inhibitor of glycogen phosphorylase, such as, for example, PSN-357 or FR-258900 or those as described in WO2003084922, WO2004007455, WO2005073229-31 or WO2005067932.

In one embodiment, the compound of the formula I is administered in combination with glucagon receptor antagonists such as, for example, A-770077, NNC-25-2504 or as described in WO2004100875 or WO2005065680.

In one embodiment, the compound of the formula I is administered in combination with activators of glucokinase, such as, for example, LY-2121260 (WO2004063179), PSN-105, PSN-110, GKA-50 or those as are described for example in WO2004072031 or WO2004072066 or WO2005080360.

In one embodiment, the compound of the formula I is administered in combination with an inhibitor of gluconeogenesis, such as, for example, FR-225654.

In one embodiment, the compound of the formula I is administered in combination with inhibitors of fructose-1,6-bisphosphatase (FBPase), such as, for example, CS-917.

In one embodiment, the compound of the formula I is administered in combination with modulators of glucose transporter 4 (GLUT4), such as, for example, KST-48 (D.-O. Lee et al.: Arzneim.-Forsch. Drug Res. 54 (12), 835 (2004)).

In one embodiment, the compound of the formula I is administered in combination with inhibitors of glutamine-fructose-6-phosphate amidotransferase (GFAT), as are described for example in WO2004101528.

In one embodiment, the compound of the formula I is administered in combination with inhibitors of dipeptidylpeptidase IV (DPP-IV), such as, for example, vildagliptin (LAF-237), sitagliptin (MK-0431), saxagliptin (BMS-477118), GSK-823093, PSN-9301, SYR-322, SYR-619, TA-6666, TS-021, GRC-8200, GW-825964X, or as are described in WO2003074500, WO2003106456, WO200450658, WO2005058901, WO2005012312, WO2005/012308, PCT/EP2005/007821, PCT/EP2005/008005, PCT/EP2005/008002, PCT/EP2005/008004, PCT/EP2005/008283, DE 10 2005 012874.2 or DE 10 2005 012873.4.

In one embodiment, the compound of the formula I is administered in combination with inhibitors of 11-beta-hydroxysteroid dehydrogenase 1 (11β-HSD1), such as, for example, BVT-2733 or those as are described for example in WO200190090-94, WO200343999, WO2004112782, WO200344000, WO200344009, WO2004112779, WO2004113310, WO2004103980, WO2004112784, WO2003065983, WO2003104207, WO2003104208, WO2004106294, WO2004011410, WO2004033427, WO2004041264, WO2004037251, WO2004056744, WO2004065351, WO2004089367, WO2004089380, WO2004089470-71, WO2004089896, WO2005016877, WO2005097759.

In one embodiment, the compound of the formula I is administered in combination with inhibitors of protein tyrosine phosphatase 1B (PTP1B), as are described for example in WO200119830-31, WO200117516, WO2004506446, WO2005012295, PCT/EP2005/005311, PCT/EP2005/005321, PCT/EP2005/007151, PCT/EP 2005/or DE 10 2004 060542.4.

In one embodiment, the compound of the formula I is administered in combination with modulators of the sodium-dependent glucose transporter 1 or 2 (SGLT1, SGLT2), such as, for example, KGA-2727, T-1095, SGL-0010, AVE 2268 and SAR 7226 or as are described for example in WO2004007517, WO200452903, WO200452902, PCT/EP2005/005959, WO2005085237, JP2004359630 or by A. L. Handlon in Expert Opin. Ther. Patents (2005) 15(11), 1531-1540.

In one embodiment, the compound of the formula I is administered in combination with modulators of GPR40.

In one embodiment, the compound of the formula I is administered in combination with inhibitors of hormone-sensitive lipase (HSL) and/or phospho-lipases as described for example in WO2005073199.

In one embodiment, the compound of the formula I is administered in combination with inhibitors of acetyl-CoA carboxylase (ACC), such as, for example, those as described in WO199946262, WO200372197, WO2003072197, WO2005044814.

In one embodiment, the compound of the formula I is administered in combination with an inhibitor of phosphoenolpyruvate carboxykinase (PEPCK), such as, for example, those as described in WO2004074288.

In one embodiment, the compound of the formula I is administered in combination with an inhibitor of glycogen synthase kinase 3 beta (GSK-3 beta), as described for example in US2005222220, WO2005085230, PCT/EP2005/005346, WO2003078403, WO2004022544, WO2003106410, WO2005058908, US2005038023, WO2005009997, US2005026984, WO2005000836, WO2004106343, EP1460075, WO2004014910, WO2003076442, WO2005087727 or WO2004046117.

In one embodiment, the compound of the formula I is administered in combination with an inhibitor of protein kinase C beta (PKC beta), such as, for example, ruboxistaurin.

In one embodiment, the compound of the formula I is administered in combination with an endothelin A receptor antagonist such as, for example, avosentan (SPP-301).

In one embodiment, the compound of the formula I is administered in combination with inhibitors of “I-kappaB kinase” (IKK inhibitors), as are described for example in WO2001000610, WO2001030774, WO2004022553 or WO2005097129.

In one embodiment, the compound of the formula I is administered in combination with modulators of the glucocorticoid receptor, like those described for example in WO2005090336.

In a further embodiment, the compound of the formula I is administered in combination with CART modulators (see “Cocaine-amphetamine-regulated transcript influences energy metabolism, anxiety and gastric emptying in mice” Asakawa, A. et al.: Hormone and Metabolic Research (2001), 33(9), 554-558);

NPY antagonists such as, for example, naphthalene-1-sulfonic acid {4-[(4-aminoquinazolin-2-ylamino)methyl]cyclohexylmethyl}amide hydrochloride (CGP 71683A);
peptide YY 3-36 (PYY3-36) or analogous compounds, such as, for example, CJC-1682 (PYY3-36 conjugated with human serum albumin via Cys34), or CJC-1643 (derivative of PYY3-36 which conjugates in vivo to serum albumin) or those as are described in WO2005080424;
Cannabinoid receptor 1 antagonists (such as, for example, rimonabant, SR147778, or those described for example in EP 0656354, WO 00/15609, WO 02/076949, WO2005080345, WO2005080328, WO2005080343, WO2005075450, WO2005080357, WO200170700, WO2003026647-48, WO200302776, WO2003040107, WO2003007887, WO2003027069, U.S. Pat. No. 6,509,367, WO200132663, WO2003086288, WO2003087037, WO2004048317, WO2004058145, WO2003084930, WO2003084943, WO2004058744, WO2004013120, WO2004029204, WO2004035566, WO2004058249, WO2004058255, WO2004058727, WO2004069838, US20040214837, US20040214855, US20040214856, WO2004096209, WO2004096763, WO2004096794, WO2005000809, WO2004099157, US20040266845, WO2004110453, WO2004108728, WO2004000817, WO2005000820, US20050009870, WO200500974, WO2004111033-34, WO200411038-39, WO2005016286, WO2005007111, WO2005007628, US20050054679, WO2005027837, WO2005028456, WO2005063761-62, WO2005061509, WO2005077897);
MC4 agonists (e.g. 1-amino-1,2,3,4-tetrahydronaphthalene-2-carboxylic acid [2-(3a-benzyl-2-methyl-3-oxo-2,3,3a,4,6,7-hexahydropyrazolo[4,3-c]pyridin-5-yl)-1-(4-chlorophenyl)-2-oxoethyl]amide; (WO 01/91752)) or LB53280, LB53279, LB53278 or THIQ, MB243, RY764, CHIR-785, PT-141 or those as are described in WO2005060985, WO2005009950, WO2004087159, WO2004078717, WO2004078716, WO2004024720, US20050124652, WO2005051391, WO2004112793, WOUS20050222014, US20050176728, US20050164914, US20050124636, US20050130988, US20040167201, WO2004005324, WO2004037797, WO2005042516, WO2005040109, WO2005030797, US20040224901, WO200501921, WO200509184, WO2005000339, EP1460069, WO2005047253, WO2005047251, EP1538159, WO2004072076, WO2004072077;
orexin receptor antagonists (e.g. 1-(2-methylbenzoxazol-6-yl)-3-[1,5]naphthyridin-4-ylurea hydrochloride (SB-334867-A) or those as are described for example in WO200196302, WO200185693, WO2004085403, WO2005075458);
histamine H3 receptor agonists (e.g. 3-cyclohexyl-1-(4,4-dimethyl-1,4,6,7-tetrahydroimidazo[4,5-c]pyridin-5-yl)propan-1-one oxalic acid salt (WO 00/63208) or those as are described in WO200064884, WO2005082893);
CRF antagonists (e.g. [2-methyl-9-(2,4,6-trimethylphenyl)-9H-1,3,9-triazafluoren-4-yl]dipropylamine (WO 00/66585));
CRF BP antagonists (e.g. urocortin);
urocortin agonists;
β3 agonists (such as, for example, 1-(4-chloro-3-methanesulfonylmethylphenyl)-2-[2-(2,3-dimethyl-1H-indol-6-yloxy)ethylamino]ethanol hydrochloride (WO 01/83451);
MSH (melanocyte-stimulating hormone) agonists;
MCH (melanin-concentrating hormone) receptor antagonists (such as, for example, NBI-845, A-761, A-665798, A-798, ATC-0175, T-226296, T-71, GW-803430 or compounds such as are described in WO 2003/15769, WO2005085200, WO2005019240, WO2004011438, WO2004012648, WO2003015769, WO2004072025, WO2005070898, WO2005070925, WO2004039780, WO2003033476, WO2002006245, WO2002002744, WO2003004027, FR2868780);
CCK-A agonists (such as, for example, {2-[4-(4-chloro-2,5-dimethoxyphenyl)-5-(2-cyclohexylethyl)thiazol-2-ylcarbamoyl]-5,7-dimethylindol-1-yl}acetic acid trifluoroacetic acid salt (WO 99/15525), SR-146131 (WO 0244150) or SSR-125180);
serotonin reuptake inhibitors (e.g. dexfenfluramine);
mixed serotoninergic and noradrenergic compounds (e.g. WO 00/71549);
5-HT receptor agonists, e.g. 1-(3-ethylbenzofuran-7-yl)piperazine oxalic acid salt (WO 01/09111);
5-HT2C receptor agonists (such as, for example, APD-356 or BVT-933 or those as are described in WO200077010, WO20077001-02, WO2005019180, WO2003064423, WO200242304, WO2005082859);
5-HT6 receptor antagonists as are described in WO2005058858;
bombesin receptor agonists (BRS-3 agonists);
galanin receptor antagonists;
growth hormone (e.g. human growth hormone or AOD-9604);
growth hormone-releasing compounds (tertiary butyl 6-benzyloxy-1-(2-diisopropylaminoethylcarbamoyl)-3,4-dihydro-1H-isoquinoline-2-carboxylate (WO 01/85695));
growth hormone secretagogue receptor antagonists (ghrelin antagonists) such as, for example, A-778193 or those as are described in WO2005030734;
TRH agonists (see, for example, EP 0 462 884);
uncoupling protein 2 or 3 modulators;
leptin agonists (see, for example, Lee, Daniel W.; Leinung, Matthew C.; Rozhavskaya-Arena, Marina; Grasso, Patricia. Leptin agonists as a potential approach to the treatment of obesity. Drugs of the Future (2001), 26(9), 873-881);
DA agonists (bromocriptine, Doprexin);
lipase/amylase inhibitors (WO 00/40569);
inhibitors of diacylglycerol O-acyltransferases (DGATs) as described for example in US2004/0224997, WO2004094618, WO200058491, WO2005044250, WO2005072740, JP2005206492, WO2005013907;
inhibitors of fatty acid synthase (FAS) such as, for example, C75 or those as described in WO2004005277;
oxyntomodulin;
oleoyl-estrone
or thyroid hormone receptor agonists such as, for example: KB-2115 or those as described in WO20058279, WO200172692, WO200194293, WO2003084915, WO2004018421, WO2005092316.

In one embodiment of the invention, the further active ingredient is leptin; see, for example, “Perspectives in the therapeutic use of leptin”, Salvador, Javier; Gomez-Ambrosi, Javier; Fruhbeck, Gema, Expert Opinion on Pharmacotherapy (2001), 2(10), 1615-1622.

In one embodiment, the further active ingredient is dexamphetamine or amphetamine.

In one embodiment, the further active ingredient is fenfluramine or dexfenfluramine.

In another embodiment, the further active ingredient is sibutramine.

In one embodiment, the further active ingredient is mazindole or phentermine.

In one embodiment, the compound of the formula I is administered in combination with bulking agents, preferably insoluble bulking agents (see, for example, Carob/Caromax® (Zunft H J; et al., Carob pulp preparation for treatment of hypercholesterolemia, ADVANCES IN THERAPY (2001 September-October), 18(5), 230-6). Caromax is a carob-containing product from Nutrinova, Nutrition Specialties & Food Ingredients GmbH, Industriepark Höchst, 65926 Frankfurt/Main)). Combination with Caromax is possible in one preparation or by separate administration of compounds of the formula I and Caromax®. Caromax® can in this connection also be administered in the form of food products such as, for example, in bakery products or muesli bars.

It will be understood that every suitable combination of the compounds of the invention with one or more of the aforementioned compounds and optionally one or more further pharmacologically active substances will be regarded as falling within the protection conferred by the present invention.

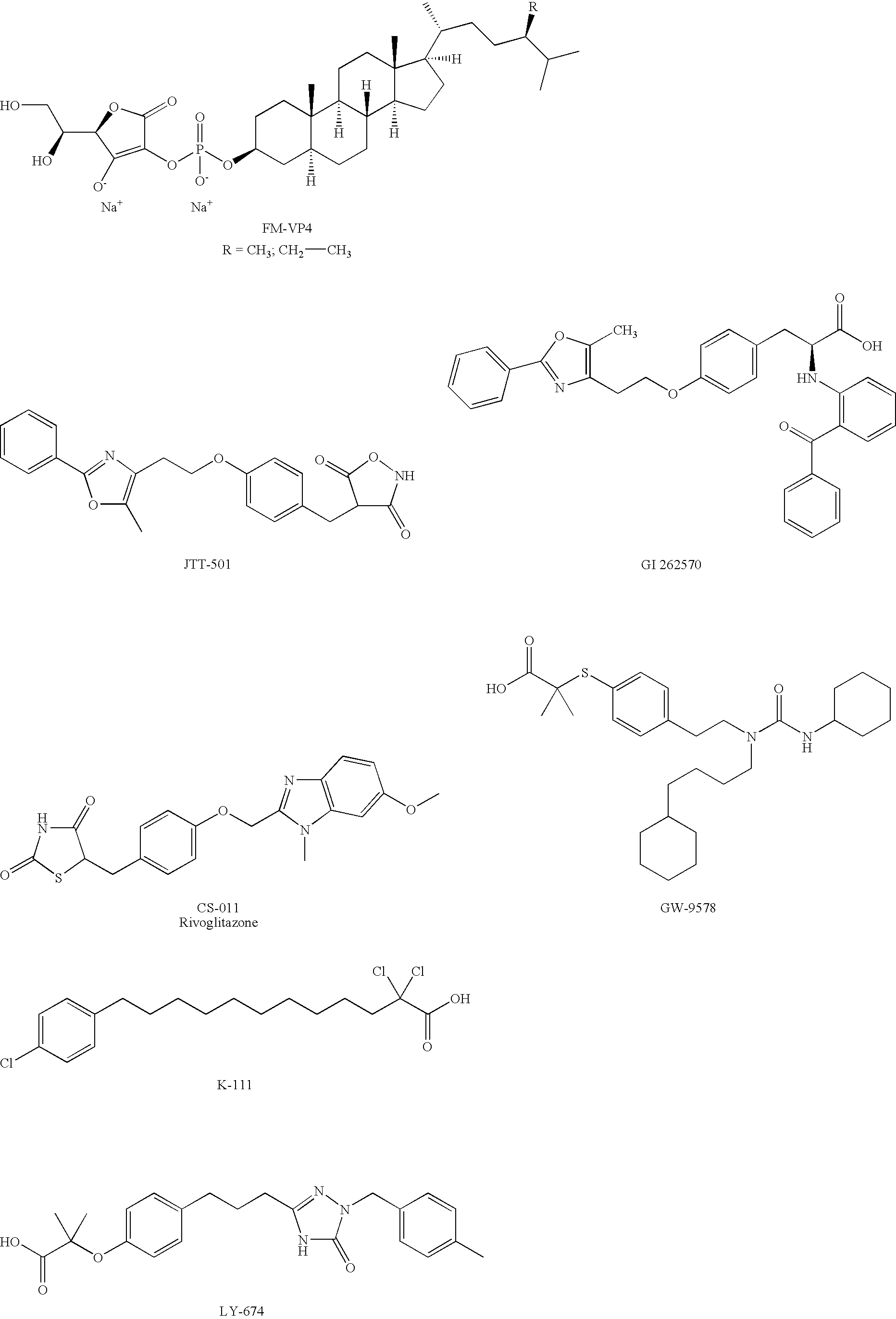

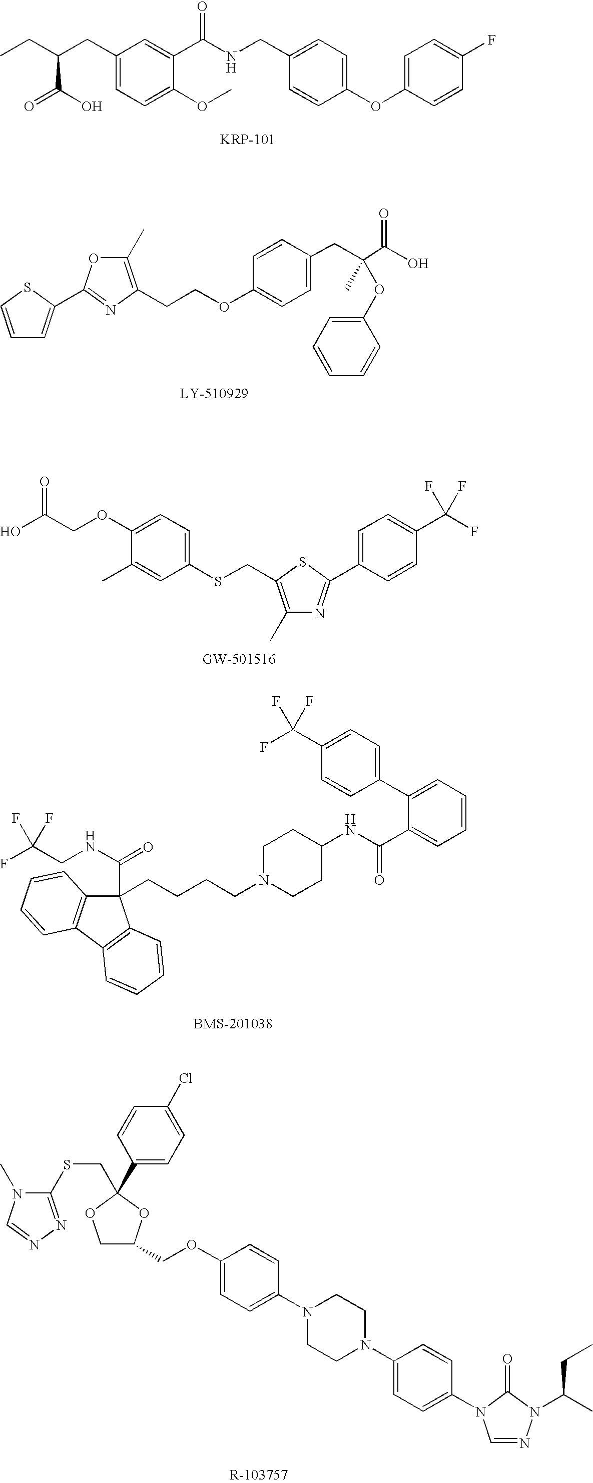

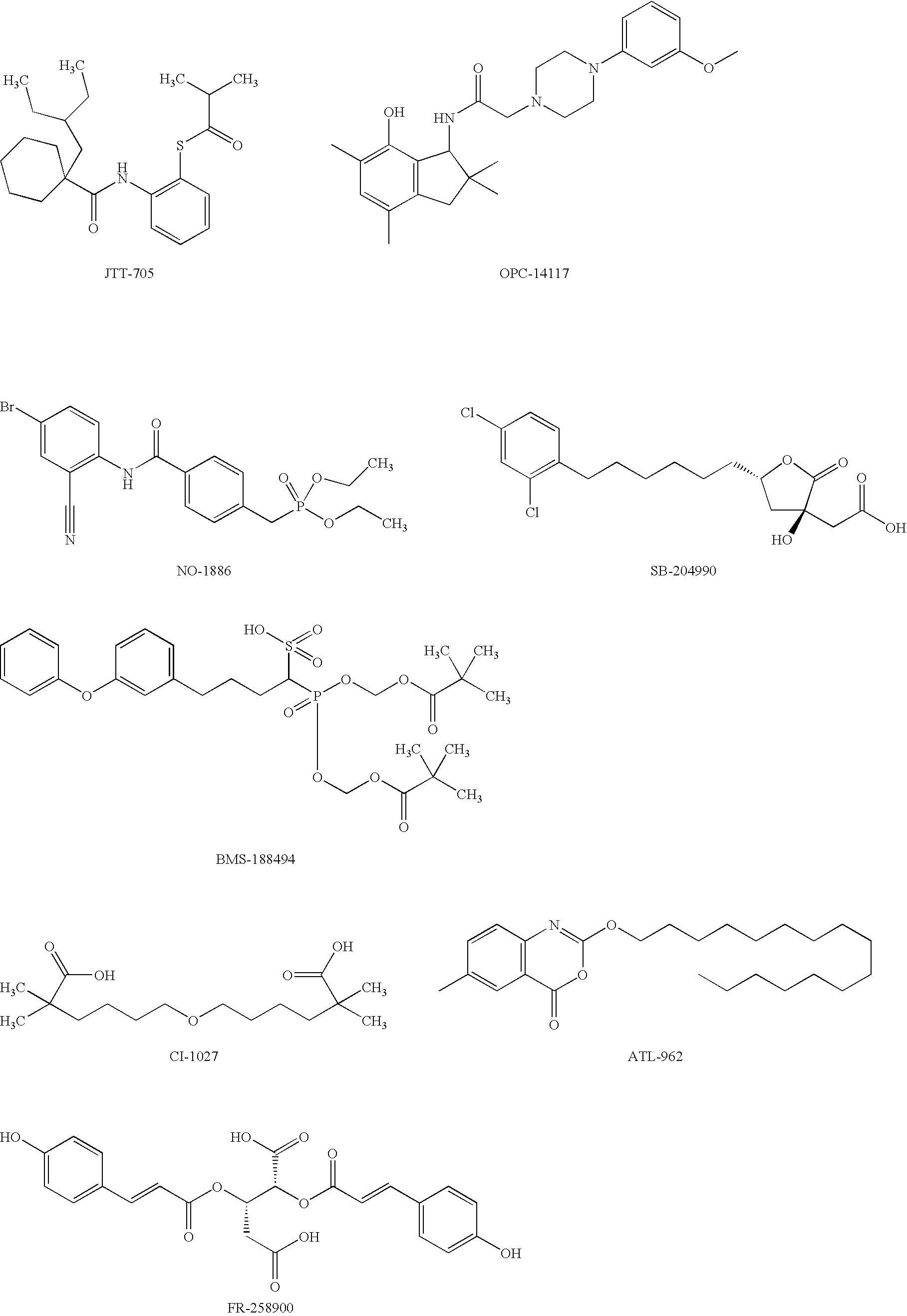

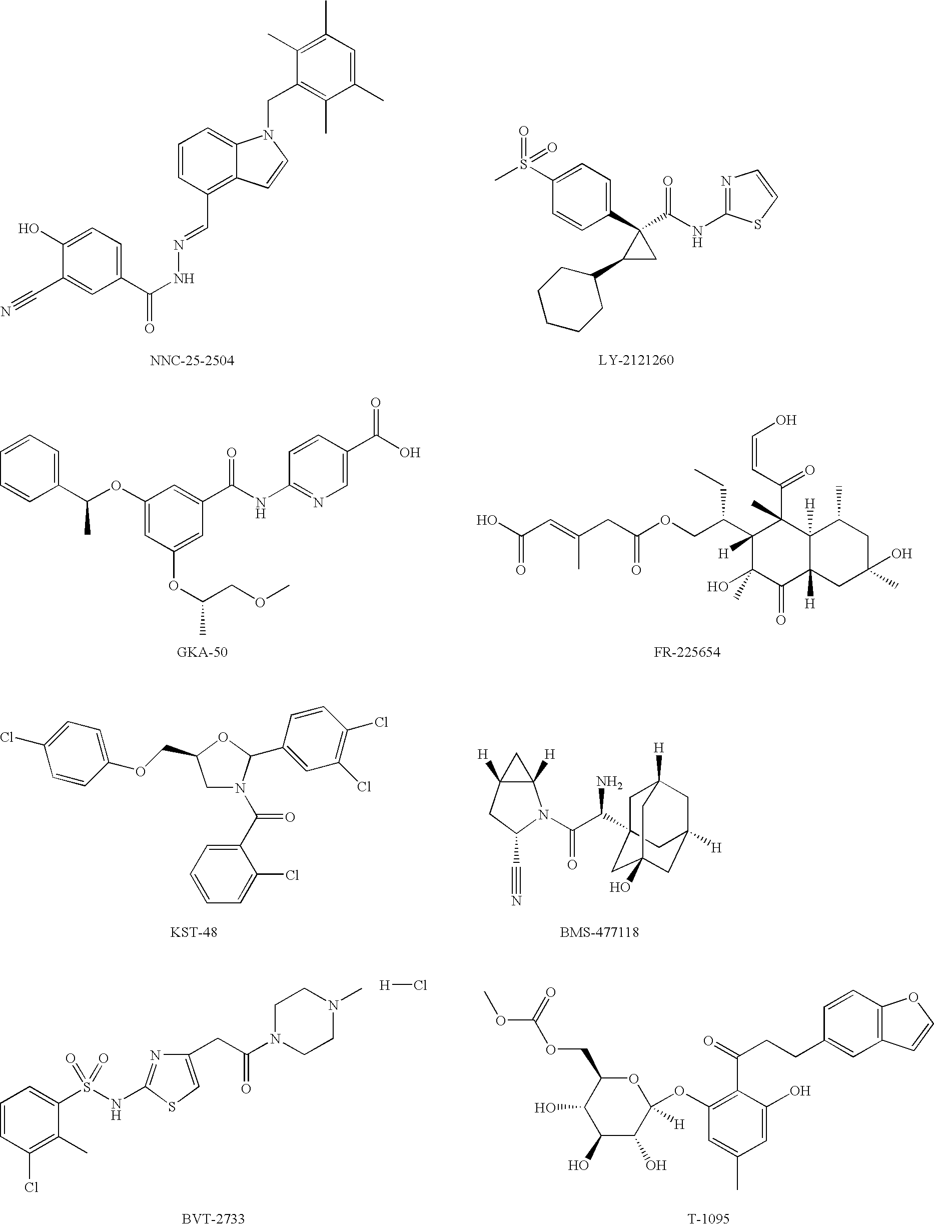

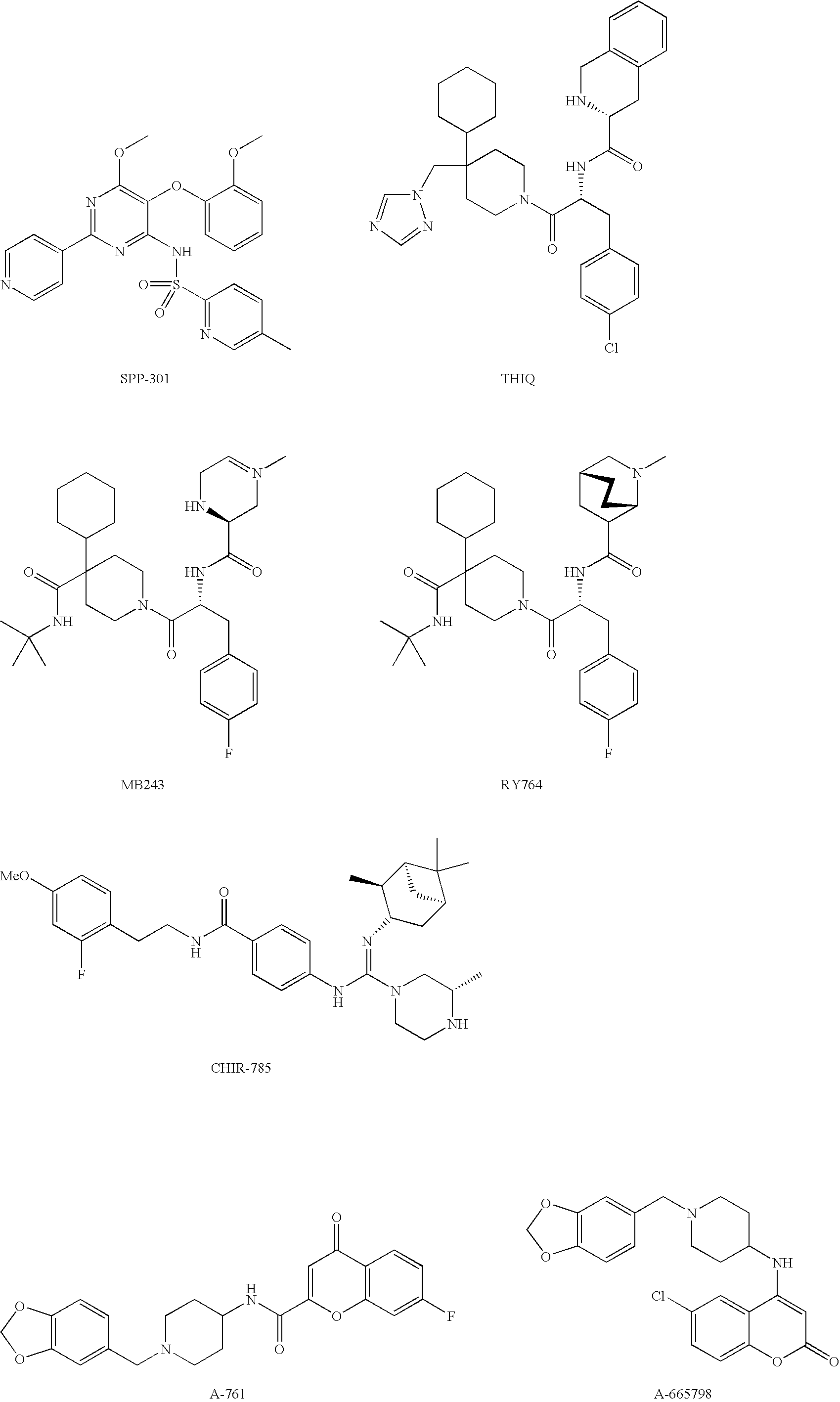

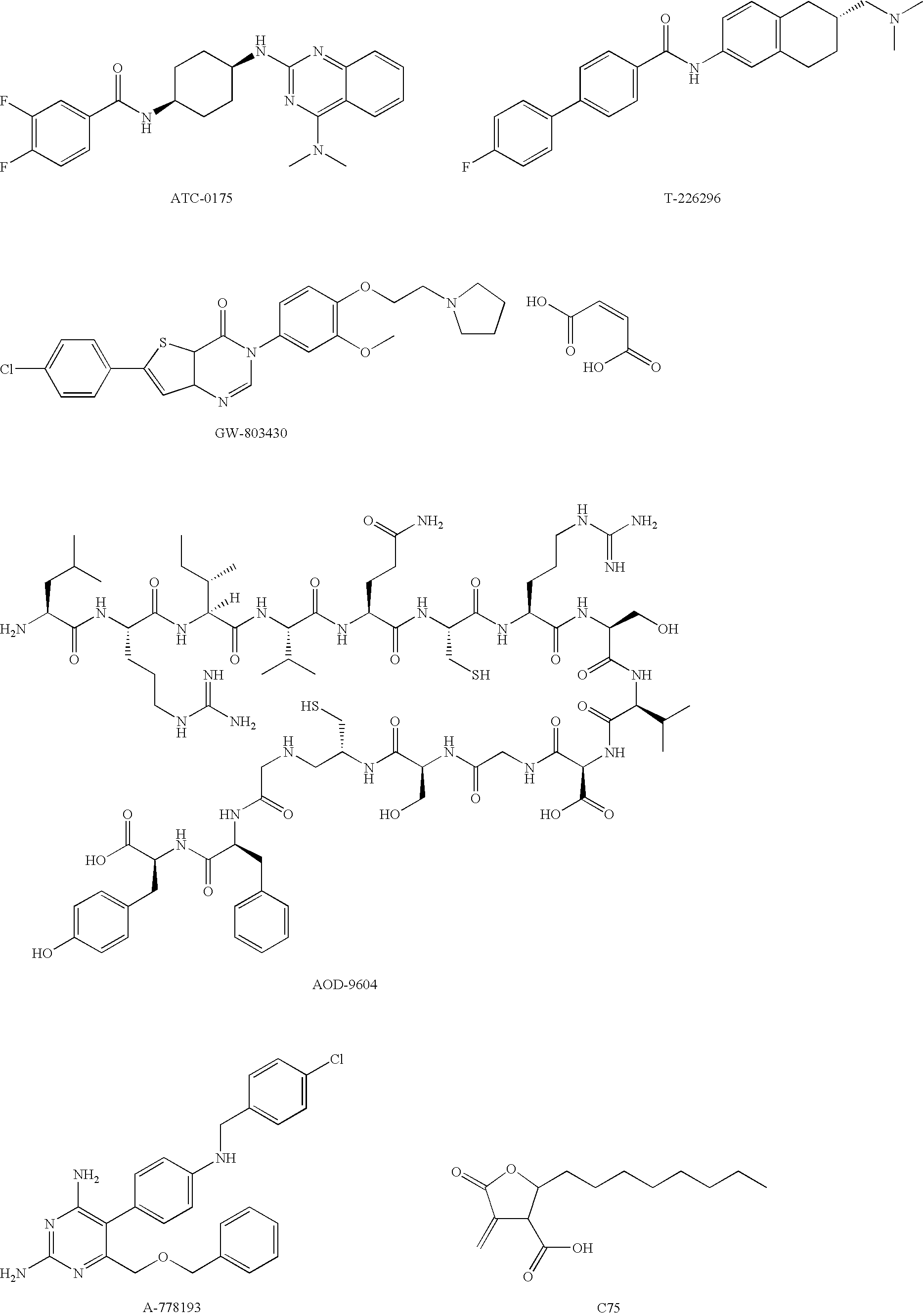

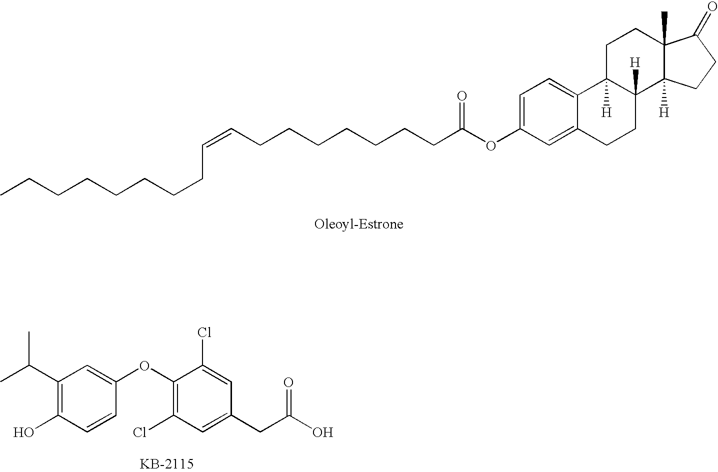

The invention further relates both to stereoisomer mixtures of the formula I and to the pure stereoisomers of the formula I, and to diastereomer mixtures of the formula I and to the pure diastereomers. The mixtures are separated by chromatographic means.

Racemic and enantiopure compounds of the formula I with the following structure are preferred:

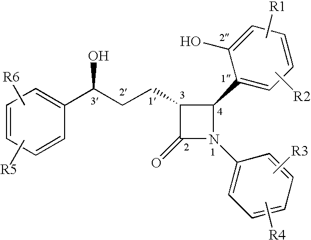

Amino protective groups which are used are preferably the benzyloxycarbonyl (Z) group which can be eliminated by catalytic hydrogenation, the 2-(3,5-dimethyloxyphenyl)propyl(2)oxycarbonyl (Ddz) or trityl (Trt) group which can be eliminated by weak acids, the t-butylcarbamate (BOC) group which can be eliminated by acids such as 3M hydrochloric acid, and the 9-fluorenylmethyloxycarbonyl (Fmoc) group which can be eliminated by secondary amines.

The invention further relates to a process for preparing diphenylazetidinone derivatives of the formula I.

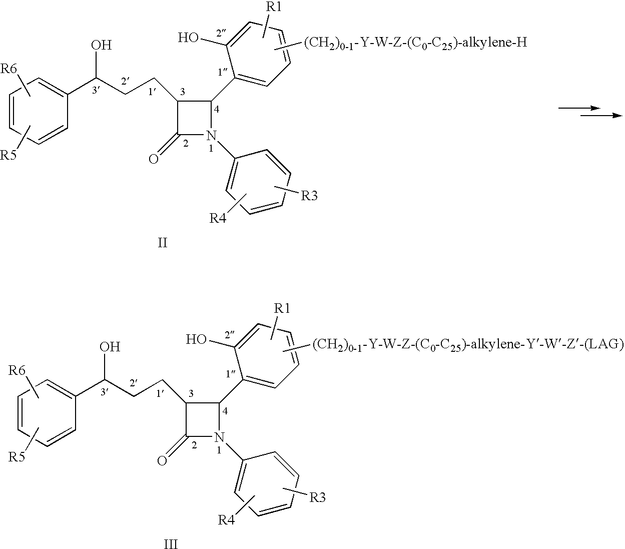

Y, W, Z, Y′, W′, Z′ may be independently of one another —NH—, —O—, —(C═O), phenyl, cycloalkyl, heterocycloalkyl or a bond, and LAG may have the meanings as described hereinbefore.

The linkage of —(CH₂)₀₋₁—Y—W-Z-(C₀-C₂₅)-alkylene-H in compound II may alternatively also be on one of the other two phenyl rings.

The process for preparing the compounds of the formula I comprises for example reacting an activated carbamate which is protected in a suitable way, or an alpha, omega diol which is protected in a suitable way, or an alpha, omega halo alcohol which is protected in a suitable way, or an alkylating agent with an amine or a hydroxyl compound of the formula II, where the hydroxyl function in the 3′ or 2″ position may be protected in a suitable way. After elimination of the protective group which may be present, the compound of the formula II can be linked in a further step with the LAG group, e.g. to form sulfuric acid monoamides.

The following examples serve to illustrate the invention in more detail without restricting the latter to the products and embodiments described in the examples.

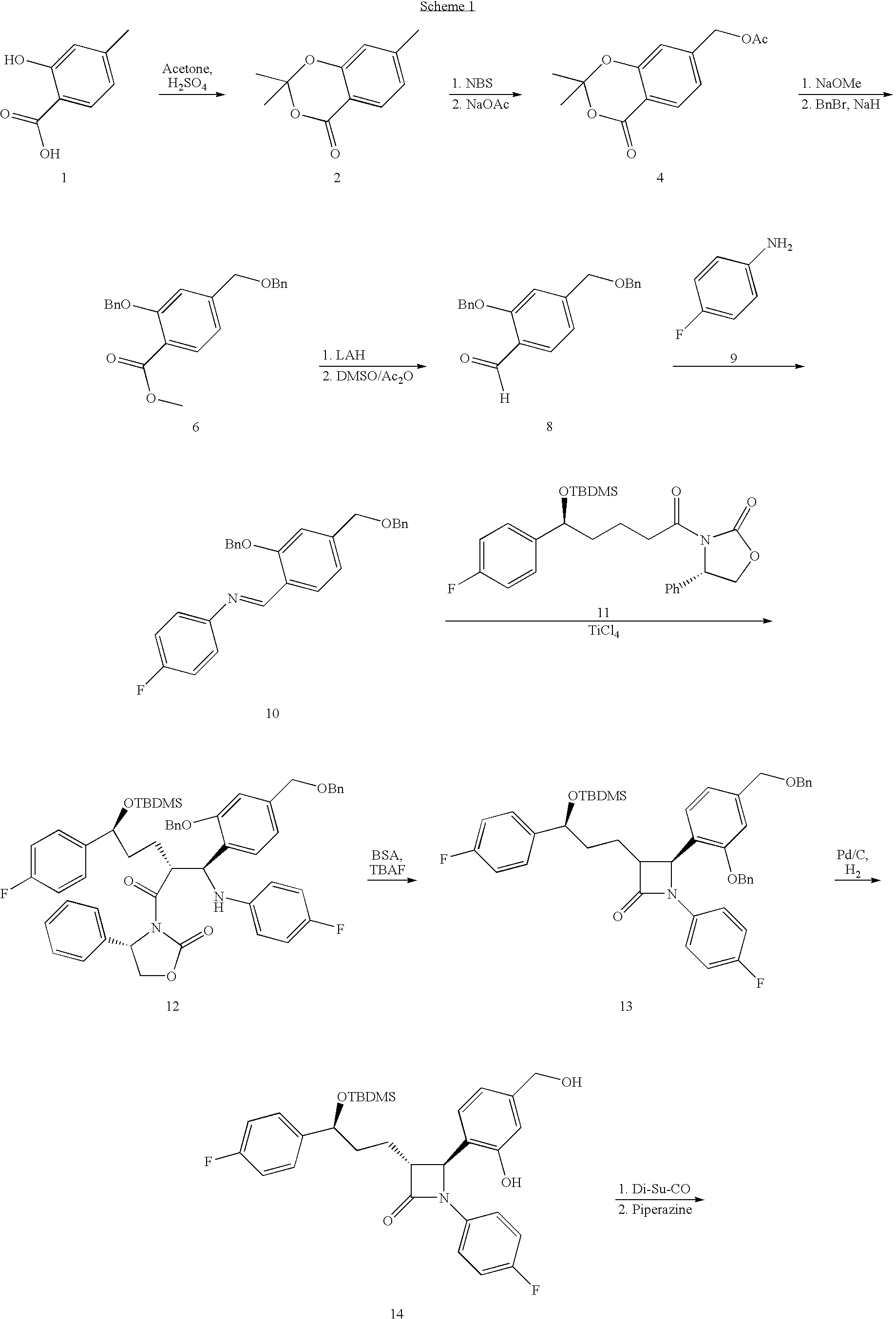

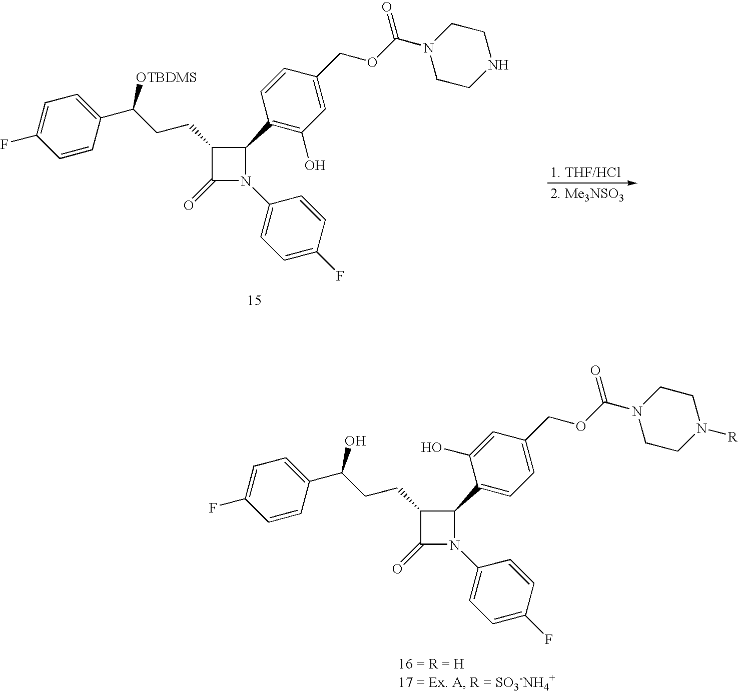

### 2,2,7-Trimethylbenzo[1,3]dioxin-4-one 2

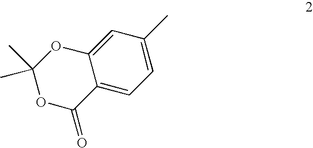

200 g (1.31 mol) of 4-methylsalicylic acid 1 are dissolved in 2 l of acetone and 400 ml of acetic anhydride. Under an argon atmosphere, 50 ml of conc. sulfuric acid are added dropwise at 0° C. The reaction solution is stirred at 0° C. for 90 minutes. Then about 1 l of acetate is distilled out, and the residue is mixed with 1 l of n-heptane/ethyl:acetate (4:1) and 1 l of water. The aqueous phase is separated off and extracted once more with 1 l of n-heptane/ethyl acetate (4:1). The collected organic phase is washed twice with 500 ml of 10% strength sodium hydroxide solution, filtered through 250 ml of silica gel and concentrated. The oily residue is dissolved in a little n-heptane and seeded with seed crystals. 137.5 g of crystalline acetonide 2 are obtained.

### 7-Bromomethyl-2,2-dimethylbenzo[1,3]dioxin-4-one 3

100 g (0.52 mol) of acetonide 2 are dissolved in 1 l of tetrachloromethane. Addition of 6 g of benzoyl peroxide is followed by boiling under reflux for 2 hours. This is followed by cooling to room temperature and dilution with 500 ml of n-heptane/ethyl acetate (3:1). This solution is filtered through silica gel and concentrated not quite to dryness (about 50 ml solvent residue). This solution is stored at −30° C. overnight. The precipitated product is filtered off with suction, and 69.5 g of crystalline benzyl bromide 3, which is still contaminated with a little peroxide, are obtained.

### 2,2-Dimethyl-4-oxo-4H-benzo[1,3]dioxin-7-ylmethyl acetate 4

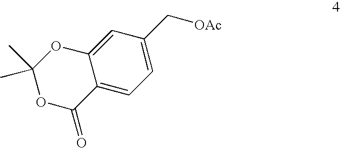

55 g (0.20 mol) of benzyl bromide 3 are dissolved in 550 ml of DMF. After addition of 30 g (0.37 mol) of sodium acetate, the mixture is left to stand overnight. For workup, 700 ml of n-heptane/ethyl acetate (2:1) and 500 ml of water are added. The aqueous phase is separated off, and the organic phase is washed twice with aqueous NaCl solution, filtered through silica gel and concentrated. The residue is purified by flash chromatography (n-heptane/ethyl acetate 4:1 to 2:1), and 22.9 g of amorphous product 4 are obtained.

### Methyl 2-hydroxy-4-hydroxymethylbenzoate 5

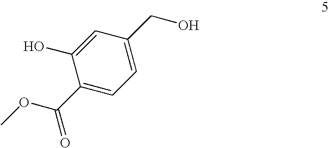

30 g (0.12 mol) of compound 4 are suspended in 100 ml of methanol. While stirring, 300 ml of 1 M NaOMe/MeOH solution are added dropwise, during which a clear solution is briefly formed. After stirring for 30 minutes, the solution is acidified with 320 ml of 1 M HCl/MeOH (the solution becomes colorless), and the resulting NaCl is removed on a little silica gel. The residue after concentration is suspended in 100 ml of n-heptane/ethyl acetate (2:1). This solution is again filtered through silica gel and washed with the n-heptane/ethyl acetate mixture. Evaporation of the solvent results in 18.1 g of crude product 5.

### Methyl 2-benzyloxy-4-benzyloxymethylbenzoate 6

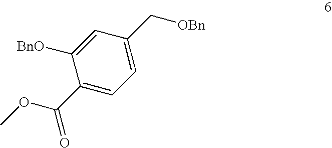

18.1 g of crude product 5 are dissolved in 360 ml of DMF and 36 ml of benzyl bromide. While cooling in an ice bath, a total of 15 g of NaH (as 55% suspension in liquid paraffin) are added in portions, and the mixture is stirred at room temperature for one hour. To destroy excess NAH and BnBr, 4 ml of methanol are cautiously added dropwise. After 15 minutes, 1 l of n-heptane/ethyl acetate (3:1) is added, and the mixture is extracted three times with water. The organic phase is filtered through silica gel and concentrated, and the residue is purified by flash chromatography (n-heptane/ethyl acetate 6:1 to 2:1). 21.4 g of perbenzylated product 6 are obtained.

### (2-Benzyloxy-4-benzyloxymethylhenyl)methanol 7

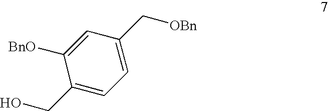

21.4 g (59.2 mmol) of methyl ester 6 are dissolved in 270 ml of THF and cooled to 0° C. A 1 M lithiumaluminum hydride solution in diethyl ether is slowly added dropwise at 0° C., and the mixture is stirred at 0° C. for 15 minutes. Excess lithiumaluminum hydride is decomposed by adding 3 ml of ethyl acetate. 4 ml of water, 4 ml of 10% strength sodium hydroxide solution and 8 ml of water are successively added cautiously in order to obtain a precipitate which can be filtered satisfactorily. The precipitate is filtered through silica gel, washed with ethyl acetate and then concentrated. 19.7 g of crude product 7 are obtained.

### 2-Benzyloxy-4-benzyloxymethylbenzaldehyde 8

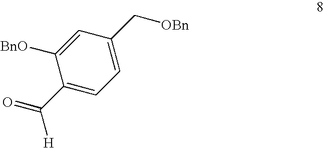

19.7 g of crude product 7 are dissolved in 300 ml of DMSO and 150 ml of acetic anhydride and left to stand at room temperature for 18 hours. This reaction solution is then diluted with 500 ml of n-heptane/ethyl acetate and washed three times with saturated NaCl solution, filtered through silica gel and concentrated. Remaining acetic anhydride is evaporated off with toluene, and 19.2 g of aldehyde 8 are obtained as a crude product.

### (2-Benzyloxy-4-benzyloxymethylbenzylidene)-(4-fluorophenyl)amine 10

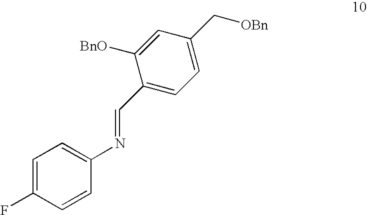

19.2 g of aldehyde 8 and 10 ml (103 mmol) of p-fluoroaniline 9 (Fluka) are boiled with 300 ml of toluene with a water trap for 2 hours, during which about 100 ml of toluene are distilled out. The remaining toluene is concentrated in a rotary evaporator, and the residue is purified by flash chromatography (n-heptane/ethyl acetate 2:1+1% triethylamine), and 25 g of imine 10 are obtained.

### 3-[2-[(2-Benzyloxy-4-benzyloxymethylphenyl)-(4-fluorophenylamino)methyl]-5-(tert-butyldimethylsilanyloxy)-5-(4-fluorophenyl)pentanoyl]-4-phenyloxazolidin-2-one 12

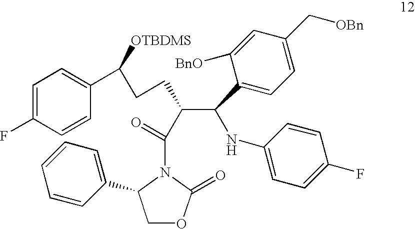

5.0 g (10.6 mmol) of oxazolidinone 11 are dissolved together with 2.1 ml of diisopropylethylamine in 50 ml of methylene chloride and cooled under argon to 0° C. 8.8 ml of a 1 M TiCl₄/methylene chloride solution are slowly added dropwise to this solution. The mixture is warmed to 20° C. for 5 minutes and then cooled to −30° C. At −30° C., a solution of 5.4 g (12.7 mmol) of imine 10 in 15 ml of methylene chloride is added dropwise, and the mixture is stirred at −30° C. for 30 minutes. The reaction solution is extracted with 100 ml of water. The organic phase is filtered through 100 ml of silica gel. The aqueous phase is again extracted with 80 ml of n-heptane/ethyl:acetate (2:1), and the organic phase is used to wash the silica gel of the first filtration. The organic phase is concentrated and purified by flash chromatography (n-heptane/ethyl acetate 4:1 to 2:1). 4.34 g of product 12 are obtained.

### 4-(2-Benzyloxy-4-benzyloxymethylphenyl)-3-[3-(tert-butyldimethylsilanyloxy)-3-(4-fluorophenyl)propyl]-1-(4-fluorophenyl)azetidin-2-one 13

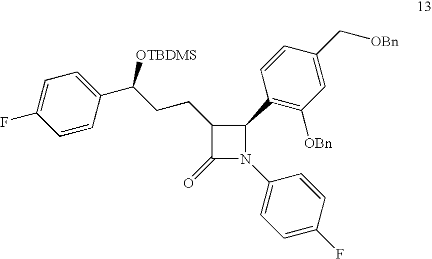

4.34 g (4.8 mmol) of compound 12 are dissolved in 60 ml of toluene. 3.4 ml of bistrimethylsilylacetamide (BSA) are added dropwise, and the mixture is cooled to 0° C. After addition of 1.7 ml of 1 M tetra-butylammonium fluoride (TBAF) in THF, the mixture is allowed to warm to room temperature and is stirred at room temperature for one hour. The reaction solution is filtered through silica gel and washed with ethyl acetate. After removal of the solvent by distillation, the residue is purified by flash chromatography (n-heptane/ethyl acetate 4:1 to 2:1), and 2.27 g of beta-lactan 13 are obtained.

### 1-(4-Fluorophenyl)-3-[3-(4-fluorophenyl)-3-hydroxypropyl]-4-(2-hydroxy-4-hydroxymethylphenyl)azetidin-2-one 14

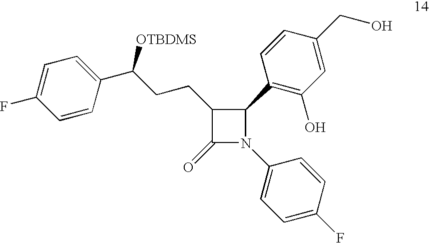

2.25 g (3.07 mmol) of lactam 13 are dissolved in 40 ml of ethyl acetate and hydrogenated with 1 g of 10% Pd on activated carbon under 6 bar of hydrogen for 3 hours. The palladium/activated carbon is removed on a little silica gel, and the residue is purified by flash chromatography (n-heptane/ethyl acetate 4:1 to 2:1). 640 mg of product 14 are obtained.

### 4-[3-[3-(tert-Butyldimethylsilanyloxy)-3-(4-fluorophenyl)propyl]-1-(4-fluorophenyl)-4-oxoazetidin-2-yl]-3-hydroxybenzylpiperazine-1-carbonate 15

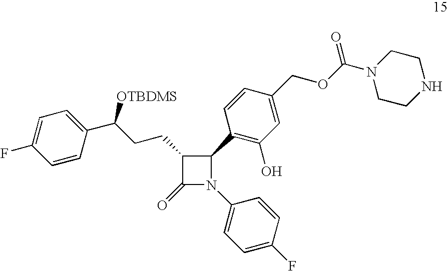

690 mg (1.25 mmol) of compound 14 are dissolved in 10 ml of acetonitrile. 0.5 ml of triethylamine and 500 mg (1.9 mmol) of di-Su-CO (Fluka) are successively added, and the mixture is left to stand at room temperature for 30 minutes. The reaction solution is then added dropwise to a solution of 1 g of piperazine in 10 ml acetonitrile, and the mixture is stirred for one hour. The heterogeneous reaction solution is purified directly by flash chromatography (methylene chloride/methanol/conc. ammonia 100/7/1 then 30/5/1 then 30/10/3), and 590 mg of product 15 are obtained as a colorless amorphous solid.

### 4-{1-(4-Fluorophenyl)-3-[3-(4-fluorophenyl)-3-hydroxypropyl]-4-oxoazetidin-2-yl}-3-hydroxybenzylpiperazine-1-carbonate 16

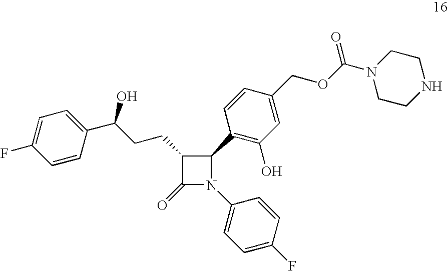

580 mg (0.87 mmol) of compound 15 are dissolved in 10 ml of THF. After addition of 5 ml of 2 N aqueous HCl, the homogeneous solution is left to stand at room temperature for 16 hours. The solution is then basified by adding a methylene chloride/methanol/conc. ammonia (30/10/30) mixture, and then concentrated. The residue is suspended in a little 30/5/1 and purified by flash chromatography (methylene chloride/methanol/conc. ammonia 30/5/1 then 30/10/3), and 400 mg of compound 16 are obtained as an amorphous solid with the molecular weight of 551.60 (C₃₀H₃₁F₂N₃O₅); MS (ESI⁺): 552.24 (M+H⁺).

### Ammonium 4-(4-{1-(4-fluorophenyl)-3-[3-(4-fluorophenyl)-3-hydroxypropyl]-4-oxoazetidin-2-yl}-3-hydroxybenzyloxycarbonyl)piperazine-1-sulfamate 17 (Example A)

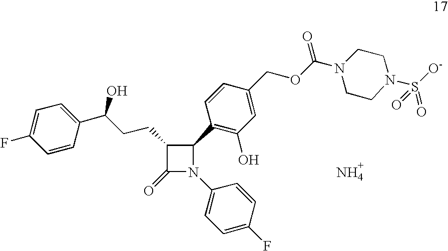

560 mg (1.0 mmol) of compound 16 are dissolved in 15 ml of methanol and cooled to 0° C. After addition of 1 g of trimethylamine-sulfur trioxide complex, the mixture is stirred at 0° C. for 2 hours. The reaction is stopped by adding 10 ml of methylene chloride/methanol/conc. ammonia 30/10/3, and the suspension is filtered through a little silica gel and washed with methylene chloride/methanol/conc. ammonia 30/10/3. The residue after concentration is purified by flash chromatography (methylene chloride/methanol/conc. ammonia 30/5/1 then 30/10/3 then 30/15/5). 580 mg of product 17 are obtained as an amorphous solid with the molecular weight of 631.20 (C₃₀H₃₁F₂N₃O₈S); MS (ESI⁻): 629.79 (M−H⁻).

### 4-(4-Bromomethyl-2-hydroxyphenyl)-3-[3-(tert-butyldimethylsilanyloxy)-3-(4-fluorophenyl)propyl]-1-(4-fluorophenyl)azetidin-2-one 18

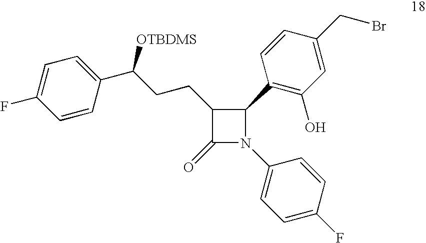

100 mg (0.18 mmol) of benzyl alcohol 14 are dissolved in 3 ml of DMF and cooled to −40° C. 180 mg of triphenylphosphine dibromide (Aldrich) are added and stirred at −40° C. for 20 minutes. The reaction solution is diluted with 20 ml of ethyl acetate and washed three times with aqueous NaCl solution, filtered through a little silica gel and concentrated, and 100 mg of crude product are obtained. This crude product is dissolved in 20 ml of THF, and 10 ml of 2 N aqueous HCl are added. After 18 hours at room temperature, the mixture is diluted with 50 ml of ethyl acetate and, after removal of the aqueous phase, washed twice with sodium bicarbonate solution, filtered through a little silica gel and concentrated, and the residue is purified by flash chromatography (n-heptane/ethyl acetate 2:1 to 1:2). 50 mg of benzyl bromide 18 are obtained.

### 1-(4-Fluorophenyl)-3-[3-(4-fluorophenyl)-3-hydroxypropyl]-4-(2-hydroxy-4-piperazin-1-ylmethylphenyl)azetidin-2-one 19

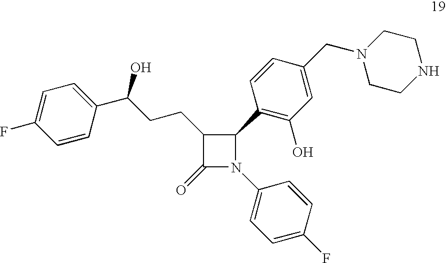

40 mg (0.08 mmol) of bromide 18 are dissolved in 2 ml of acetonitrile. Addition of 50 mg of piperazine is followed by stirring at room temperature for one hour. The suspension is concentrated and the residue is purified by flash chromatography (methylene chloride/methanol/conc. ammonia 30/5/1 then 30/10/3). 42 mg of amine 19 are obtained.

### Ammonium 4-(4-{1-(4-Fluorophenyl)-3-[3-(4-fluorophenyl)-3-hydroxypropyl]-4-oxoazetidin-2-yl}-3-hydroxybenzyl)piperazine-1-sulfamate (Example B)

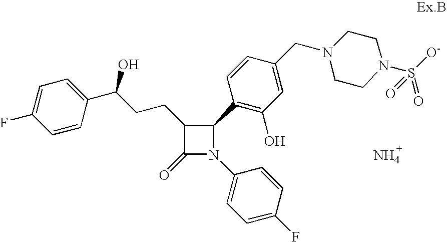

38 mg (0.075 mmol) of compound 19 are reacted in analogy to the synthesis of example A, and 30 mg of example B are obtained as an amorphous solid with the molecular weight of 587.11 (C₂₉H₃₁F₂N₃O₆S); MS (ESI⁻): 586.11 (M−H⁻).

### 4-{1-(4-Fluorophenyl)-3-[3-(4-fluorophenyl)-3-hydroxypropyl]-4-oxoazetidin-2-yl}-3-hydroxybenzyl 4-methylpiperazine-1-carbonate 20

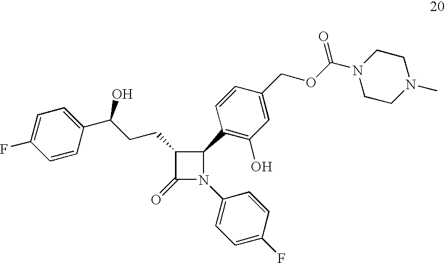

200 mg (0.36 mmol) of benzyl alcohol 14 are reacted in analogy to the synthesis of compound 15 and 16, and 90 mg of methylpiperazine 20 are obtained as an amorphous solid.

### 4-(4-{1-(4-Fluorophenyl)-3-[3-(4-fluorophenyl)-3-hydroxypropyl]-4-oxoazetidin-2-yl}-3-hydroxybenzyloxycarbonyl)-1,1-dimethylpiperazin-1-ium iodide (Example C)

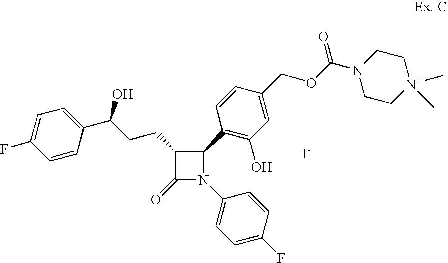

80 mg (0.14 mmol) of compound 20 are dissolved in 5 ml of toluene and heated to 80° C. 0.8 ml of methyl iodide is added dropwise to this solution, and the mixture is stirred at 80° C. for one hour. The precipitated product is filtered and washed with toluene, and 72 mg of colorless solid of the ammonium salt Ex. C are obtained with the molecular weight of 580.26 (C₃₂H₃₆F₂N₃O₅); MS (ESI): 580.12 (M⁺).

### 4-(4-{1-(4-Fluorophenyl)-3-[3-(4-fluorophenyl)-3-hydroxypropyl]-4-oxoazetidin-2-yl}-3-hydroxybenzyloxycarbonylmethyl)-1-methylpyridinium iodide (Example D)

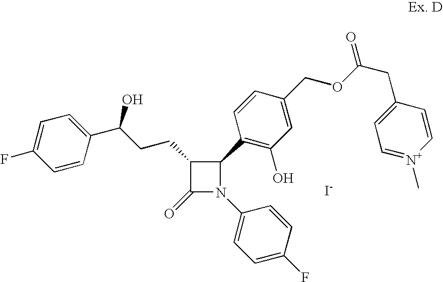

135 mg (0.24 mmol) of benzyl alcohol 14 are reacted in analogy to the synthesis of compound example C, and 53 mg of ammonium salt example D are obtained as a colorless solid with the molecular weight of 588.23 (C₃₃H₃₂F₂N₃O₅); MS (ESI): 588.36 (M⁺).

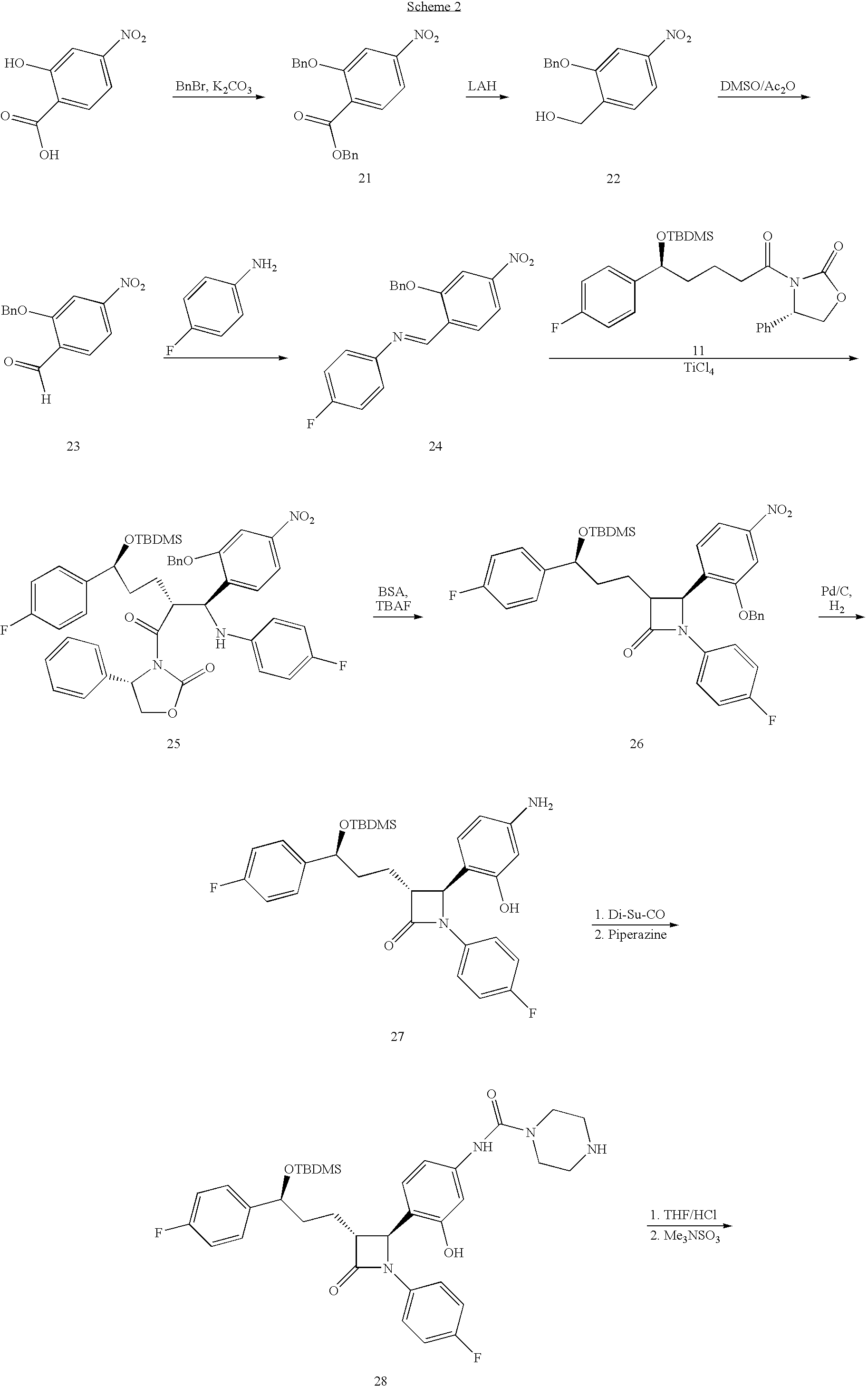

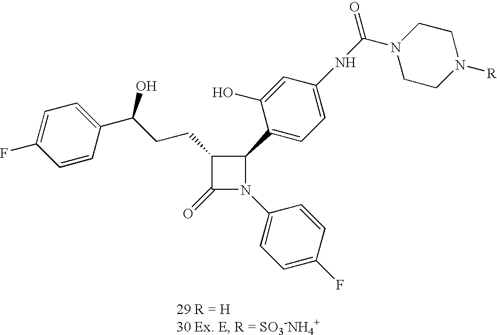

### Benzyl 2-benzyloxy-4-nitrobenzoate 21

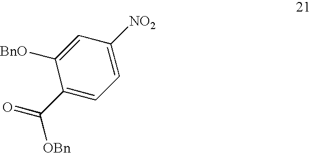

75 g (410 mmol) of 4-nitrosalicylic acid (Aldrich) are dissolved in 1 l of DMF and 190 ml of benzyl bromide. After addition of 200 g of K₂CO₃, the suspension is stirred vigorously at room temperature for 16 hours. The reaction solution is stirred with 1 l of ethyl acetate and 1 l of water for 15 minutes. The aqueous phase is separated off, and the organic phase is washed twice with saturated NaCl solution, filtered through silica gel and concentrated until the product starts to crystallize. The crystals are filtered off with suction (70 g) and washed with n-heptane/ethyl acetate (4:1). On concentration of the mother liquor, a further fraction of crystals separates out (this can be repeated two times more). In total, 132 g of crystalline product 21 are obtained.

### 2-Benzyloxy-4-nitrobenzaldehyde 23

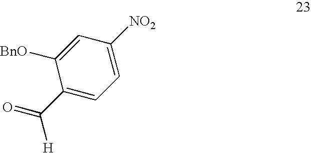

132 g (363 mmol) of nitroaromatic compound 21 are reacted in analogy to the synthesis of compounds 7 and 8, and aldehyde 23 is obtained as a colorless solid.

### (2-Benzyloxy-4-nitrobenzylidene)-(4-fluorophenyl)amine 24

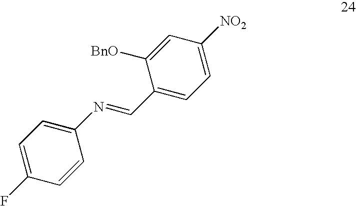

22.1 g (86 mmol) of aldehyde 23 and 12.0 g (108 mmol) of 4-fluoroaniline 9 are reacted in analogy to the synthesis of imine 10, and imine 24 is obtained as a crystalline solid.

### 4-(2-Benzyloxy-4-nitrophenyl)-3-[3-(tert-butyldimethylsilanyloxy)-3-(4-fluorophenyl)propyl]-1-(4-fluorophenyl)azetidin-2-one 26

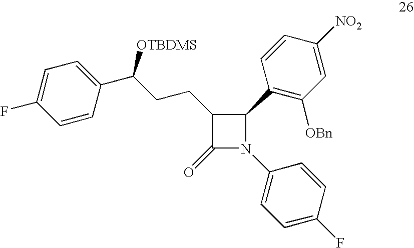

24 g of beta-lactam 26 are obtained as an amorphous solid from 27.5 g (78.5 mmol) of imine 24 and 62.2 g (132 mmol) of oxazolidinone 11 in a preparation process analogous to that for compound 12 and 13.

### 4-(4-Amino-2-benzyloxyphenyl)-3-[3-(tert-butyldimethylsilanyloxy)-3-(4-fluorophenyl)propyl]-1-(4-fluorophenyl)azetidin-2-one 27

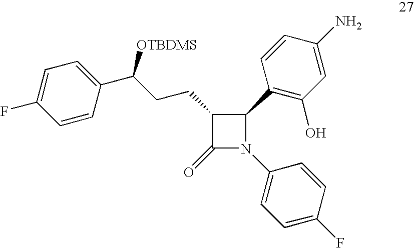

24 g (36 mmol) of 26 are reacted in analogy to the hydrogenation of compound 13, and 13.2 g of aniline 27 are obtained as an amorphous solid.

### Piperazine-1-carbonic acid {4-[3-[3-(tert-butyldimethylsilanyloxy)-3-(4-fluorophenyl)propyl]-1-(4-fluorophenyl)-4-oxoazetidin-2-yl]-3-hydroxyphenyl}amide 28

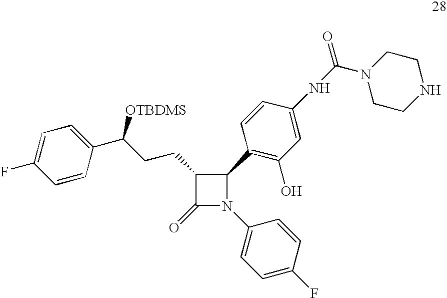

700 mg (1.3 mmol) of aniline 27 are reacted in analogy to the synthesis of amine 15, and 347 mg of 28 are obtained as an amorphous solid.

### Piperazine-1-carbonic acid (4-{1-(4-fluorophenyl)-3-[3-(4-fluorophenyl)-3-hydroxypropyl]-4-oxoazetidin-2-yl}-3-hydroxyphenyl)amide 29

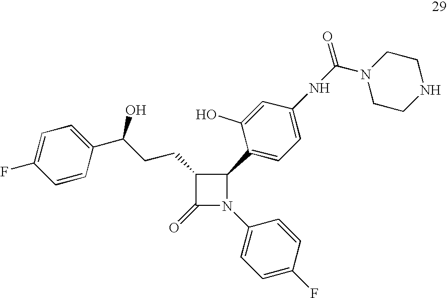

337 mg (0.52 mmol) of piperazine derivative 28 are reacted in analogy to the synthesis of compound 16, and 217 mg of 29 are obtained as an amorphous solid with the molecular weight of 536.58 (C₂₉H₃₀F₂N₄O₄); MS (ESI⁺): 537.13 (M+H⁺).

### Ammonium 4-(4-{1-(4-fluorophenyl)-3-[3-(4-fluorophenyl)-3-hydroxypropyl]-4-oxoazetidin-2-yl}-3-hydroxyphenylcarbamoyl)piperazine-1-sulfamate 30 (Example E)

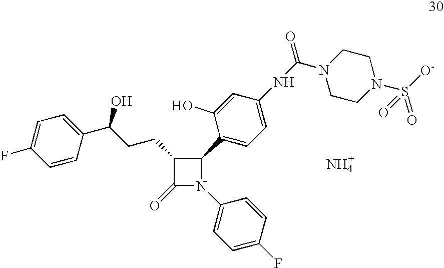

370 mg (0.69 mmol) of compound 29 are reacted in analogy to the synthesis of example A, and 343 mg of sulfamide 30 (example E) are obtained as an amorphous solid with a molecular weight of 616.18 (C₂₉H₃₀F₂N₄O₇S); MS (ESI⁺): 1233.28 (2M+H⁺).

### 4-(4-Amino-2-hydroxyphenyl)-1-(4-fluorophenyl)-3-[3-(4-fluorophenyl)-3-hydroxypropyl]azetidin-2-one 31

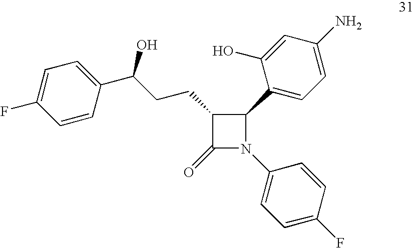

2.5 g (3.8 mmol) of compound 27 are silyl-deprotected in analogy to the synthesis of compound 16, and 1.23 g of aniline 31 are obtained as an amorphous solid.

### Ammonium (4-[1-(4-fluorophenyl)-3-[3-(4-fluorophenyl)-3-hydroxypropyl]-4-oxoazetidin-2-yl]-3-hydroxyphenyl)sulfamate (Example F)

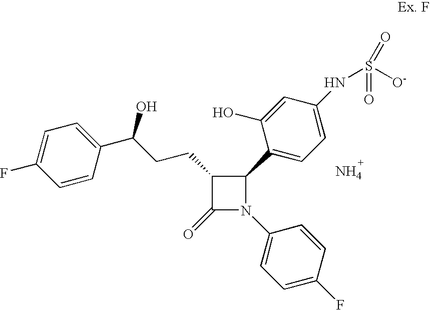

44 mg (0.10 mmol) of aniline 31 are sulfated in analogy to the synthesis of compound 30 with 120 mg of trimethylamine-sulfur dioxide complex, and 21 mg of sulfamide Ex. F are obtained as an amorphous solid with the molecular weight of 504.12 (C₂₄H₂₂F₂N₂O₆S); MS (ESI⁻): 503.25 (M−H⁻).

### 2,3,4,5,6-Pentahydroxyhexanoic acid (4-{1-(4-fluorophenyl)-3-[3-(4-fluorophenyl)-3-hydroxypropyl]-4-oxoazetidin-2-yl}-3-hydroxyphenyl)amide (Example G)

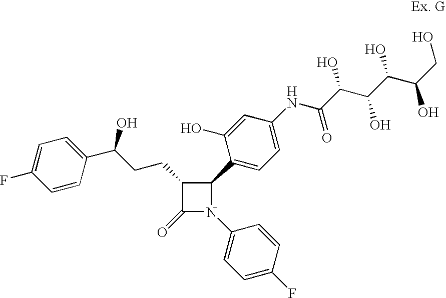

50 mg (0.12 mmol) of aniline 31 and 100 mg (0.24 mmol) of penta-O-acetylgluconic acid are dissolved in 3 ml of DMF. 150 mg of EDC and 75 mg of HOBt are added, and the mixture is stirred at room temperature for one hour. Addition of 20 ml of ethyl acetate is followed by washing three times with NaCl solution, filtering through silica gel and concentrating, and the residue is purified by flash chromatography (n-heptane/ethyl acetate 1:1 to 0:1). The resulting product (65 mg) is dissolved in 5 ml of methanol, and 0.5 ml of 1 M NaOMe/MeOH is added. After 30 minutes at room temperature, the mixture is neutralized with 0.5 M HCl/MeOH and concentrated, and the residue is purified by flash chromatography (methylene chloride/methanol/conc. ammonia 30/5/1 then 30/10/3 then 30/15/5). 40 mg of sugar derivative Ex. G are obtained as an amorphous solid with the molecular weight of 602.59 (C₃₀H₃₂F₂N₂O₉); MS (ESI⁻): 601.40 (M−H⁻).

### 1-[(4-{1-(4-Fluorophenyl)-3-[3-(4-fluorophenyl)-3-hydroxypropyl]-4-oxoazetidin-2-yl}-3-hydroxyphenylcarbamoyl)methyl]-4-aza-1-azoniabicyclo[2.2.2]octane iodide (example

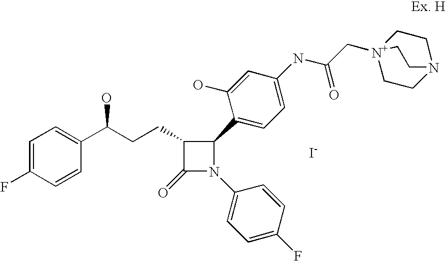

54 mg (0.13 mmol) of aniline 31 and 150 mg of iodoacetic acid are coupled with EDC/HOBt in analogy to the description for the synthesis of Ex. G, and 40 mg of iodide are obtained. This is dissolved in 5 ml of toluene and 1 ml of methylene chloride. Addition of 200 ml of DABCO is followed by stirring at 80° C. for one hour. The precipitate is filtered off with suction, and the trialkylammoniumalkyl salt Ex. H is obtained with a molecular weight of 577.26 (C₃₂H₃₅F₂N₄O₄); MS (ESI): 577.20 (M⁺).

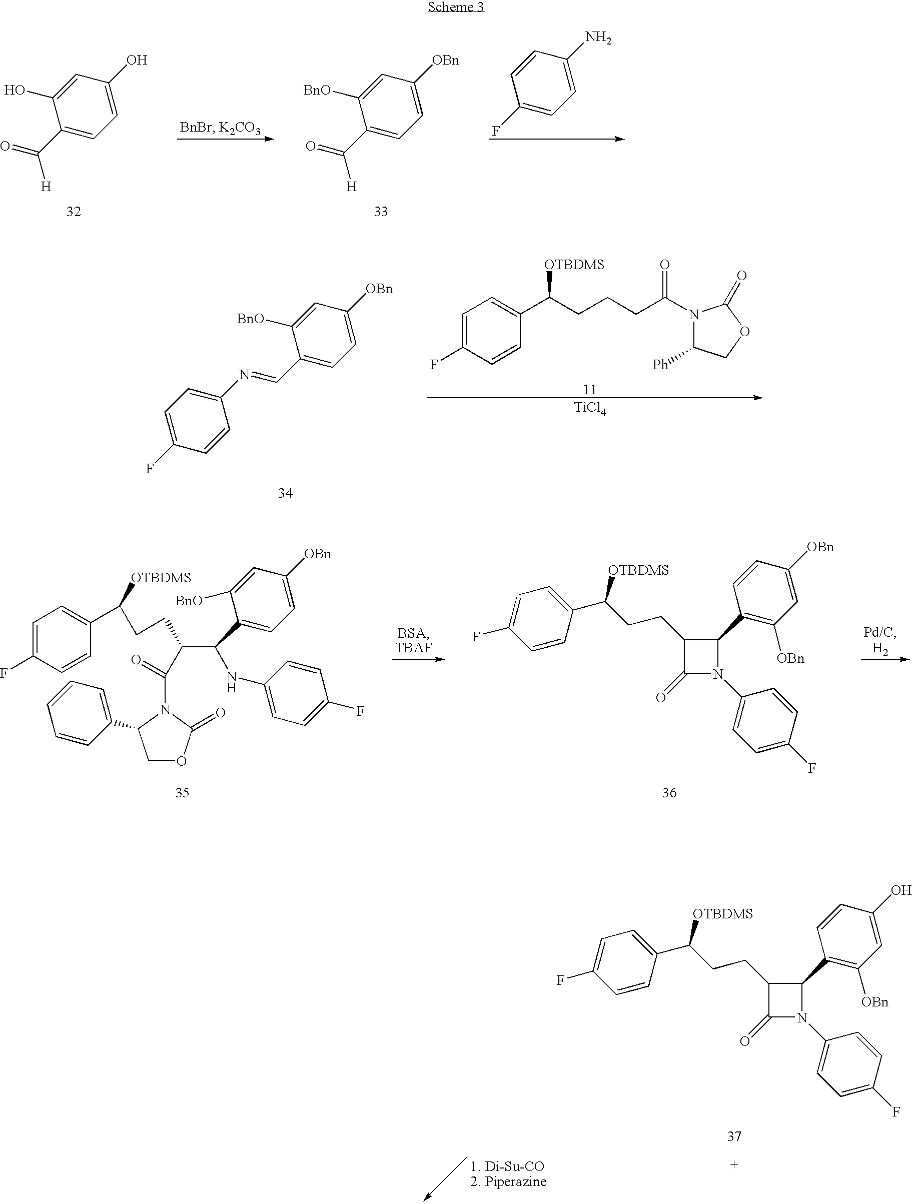

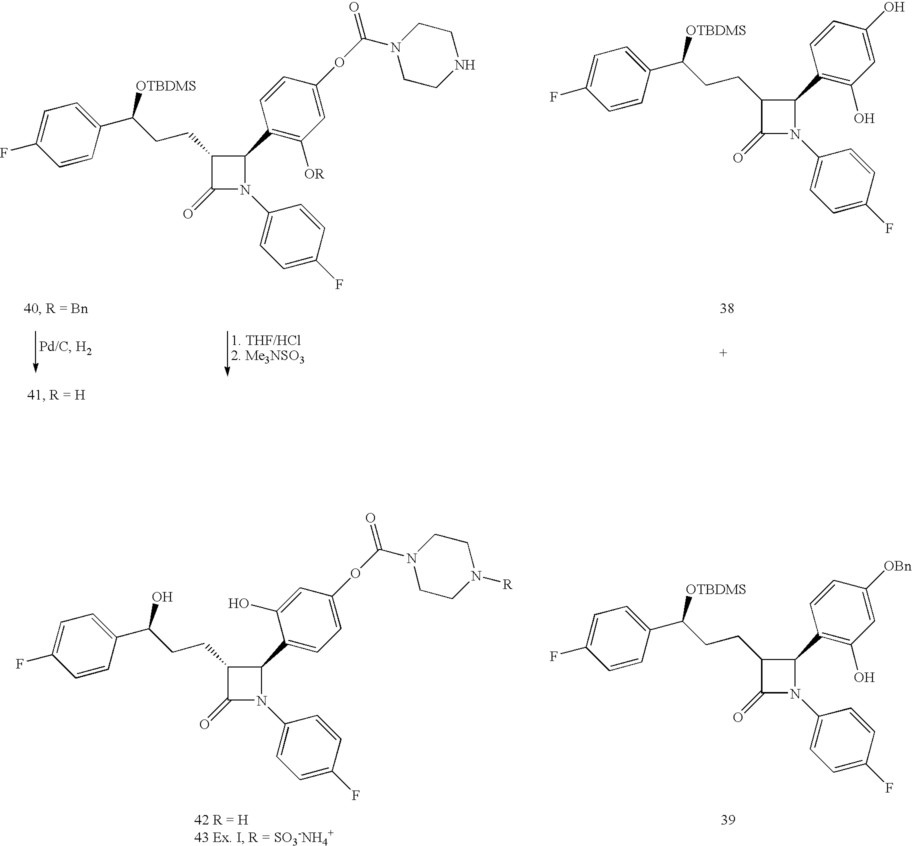

### 2,4-Dibenzyloxybenzaldehyde 33

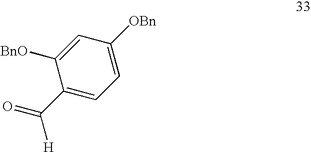

50 g (362 mmol) of 2,4-dihydroxybenzaldehyde 32 (Aldrich) are perbenzylated in 800 ml of DMF with 100 ml of benzyl bromide and 200 g of K₂CO₃ in analogy to the synthesis of 21, and 96 g of crystalline product 33 are obtained.

### (2,4-Dibenzyloxybenzylidene)-(4-fluorophenyl)amine 34

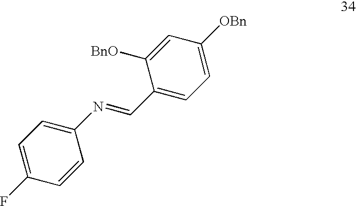

6.1 g (1.9 mmol) of aldehyde 33 are reacted with 3.0 g (2.7 mmol) of 4-fluoroaniline 9 in analogy to the synthesis of imine 10, and 6.0 g of crystalline imine 34 are obtained.

### 3-[2-[(2,4-Dibenzyloxyphenyl)-(4-fluorophenylamino)methyl]-5-(tert-butyldimethylsilanyloxy)-5-(4-fluorophenyl)pentanoyl]-4-phenyloxazolidin-2-one 35

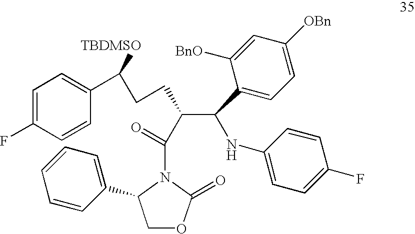

5.0 g (10.6 mmol) of oxazolidinone 11 and 8.0 g (19.4 mmol) of imine 34 are reacted in analogy to the synthesis of compound 12, and 7.5 g of imine adduct 35 are obtained as an amorphous solid.

### 3-[3-(tert-Butyldimethylsilanyloxy)-3-(4-fluorophenyl)propyl]-4-(2,4-dihydroxyphenyl)-1-(4-fluorophenyl)azetidin-2-one 36

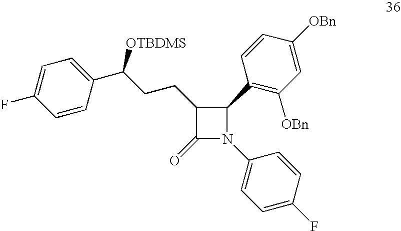

28.4 g (32.2 mmol) of 35 are cyclized in analogy to the synthesis of beta-lactam 13, and 13.1 g of lactam 36 are obtained as an amorphous solid.

### 4-(2-Benzyloxy-4-hydroxyphenyl)-3-[3-(tert-butyldimethylsilanyloxy)-3-(4-fluorophenyl)propyl]-1-(4-fluorophenyl)azetidin-2-one 37

### 3-[3-(tert-Butyldimethylsilanyloxy)-3-(4-fluorophenyl)propyl]-4-(2,4-dihydroxyphenyl)-1-(4-fluorophenyl)azetidin-2-one 38

### 4-(4-Benzyloxy-2-hydroxyphenyl)-3-[3-(tert-butyldimethylsilanyloxy)-3-(4-fluorophenyl)propyl]-1-(4-fluorophenyl)azetidin-2-one 39

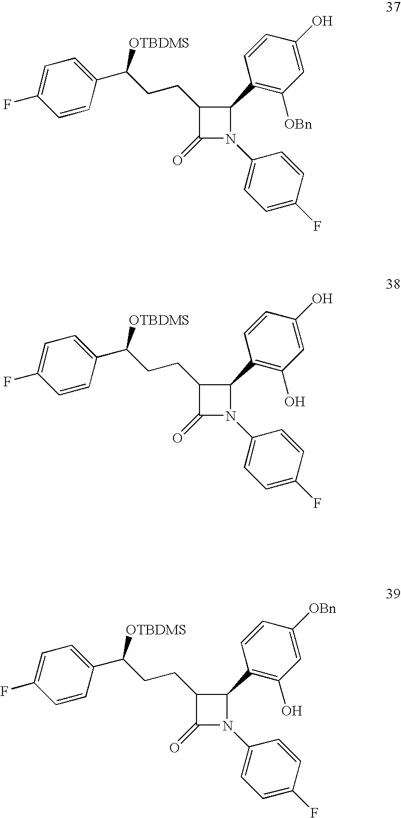

Partial hydrogenation of compound 36 affords a 5.3:1.7:1 mixture of products 37:38:39. The diphenol 38 can be obtained quantitatively by complete hydrogenation. 13.1 g of lactone 36 are dissolved in 200 ml of ethyl acetate and hydrogenated with 2 g of Pd/C (10%/Pd) under 5 bar of hydrogen for 3 hours. The product mixture is fractionated by flash chromatography (n-heptane/ethyl acetate 3:1 to 1:2). Product 39 is eluted first, then 37 and, as the most polar compound, the product 38. 5.3 g of 37, 1.7 g of 38 and 1.0 g of 39 are obtained, all as amorphous solids.

### 3-Benzyloxy-4-[3-[3-(tert-butyldimethylsilanyloxy)-3-(4-fluorophenyl)pyropyl]-1-(4-fluorophenyl)-4-oxoazetidin-2-yl]phenyl piperazine-1-carbonate 40

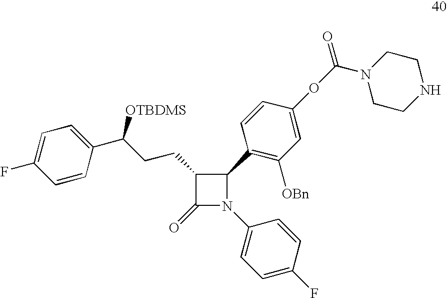

1.0 g (1.6 mmol) of phenol 37 are reacted with 1.0 g of Di-SU-CO in analogy to the synthesis of compound 15, and 387 mg of piperazine derivative 40 and 405 mg of starting material 37 (this is formed from the intermediate by attack of the piperazine on the phenolic side of the carbonyl group) are obtained.

### Piperazine-1-carbonic acid {4-[3-[3-(tert-butyldimethylsilanyloxy)-3-(4-fluorophenyl)-propyl]-1-(4-fluorophenyl)-4-oxoazetidin-2-yl]-3-hydroxyphenyl}amide 41

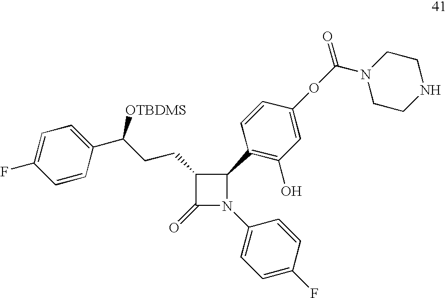

370 mg (0.50 mmol) of compound 40 are benzyl-deprotected in analogy to the hydrogenation method for compound 14, and 310 mg of piperazine derivative 41 are obtained as an amorphous solid.

### 4-{1-(4-Fluorophenyl)-3-[3-(4-fluorophenyl)-3-hydroxypropyl]-4-oxoazetidin-2-yl}-3-hydroxyphenyl piperazine-1-carbonate 42

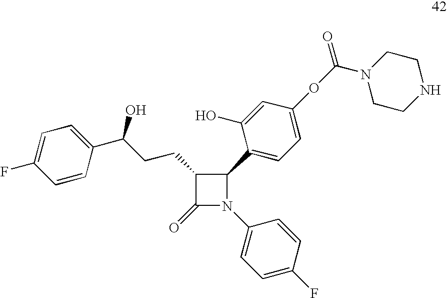

310 mg (0.48 mmol) of compound 41 are deprotected with aqueous HCl (in analogy to compound 16), and 124 mg of piperazine derivative 42 are obtained as an amorphous solid with the molecular weight of 537.57 (C₂₉H₂₉F₂N₃O₅); MS (ESI⁺): 538.18 (M+H⁺).

### Ammonium 4-(4-{1-(4-Fluorophenyl)-3-[3-(4-fluorophenyl)-3-hydroxypropyl]-4-oxoazetidin-2-yl}-3-hydroxyphenoxycarbonyl)piperazine-1-sulfamate 43 (example 1)

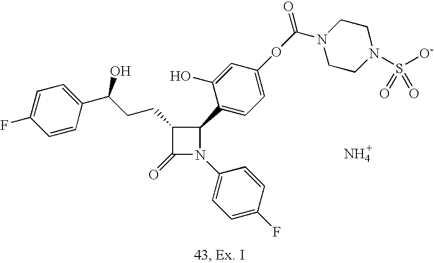

70 mg (0.13 mmol) of 42 are reacted with the SO₃-trimethylamine complex in analogy to the synthesis of compound 17. 77 mg of sulfamide derivative 43 (example 1) are obtained as an amorphous solid with the molecular weight of 617.16 (C₂₉H₂₉F₂N₃O₈S); MS (ESI⁺): 1235.05 (2M+H⁺).

### 4-{1-(4-Fluorophenyl)-3-[3-(4-fluorophenyl)-3-hydroxypropyl]-4-oxoazetidin-2-yl}-3-hydroxyphenyl 4-methylpiperazine-1-carbonate, compound 70

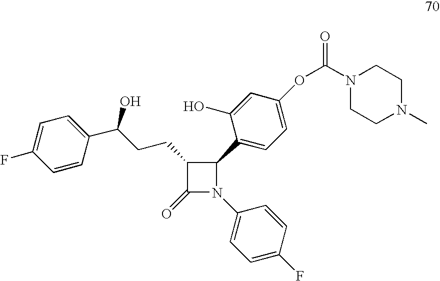

Synthesis of compound 70 is carried out in analogy to the preparation of compound 42. Methylpiperazine is used instead of piperazine. Molecular weight 551.60 (C₃₀H₃₁F₂N₃O₅); MS (ESI⁺): 1103.08 (2M+H⁺).

### 4-(4-{1-(4-Fluorophenyl)-3-[3-(4-fluorophenyl)-3-hydroxypropyl]-4-oxoazetidin-2-yl}-3-hydroxyphenoxycarbonyl)-1,1-dimethylpiperazin-1-ium iodide, Example J

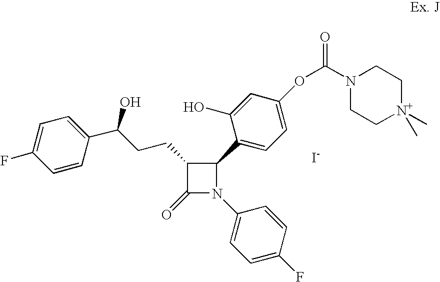

56 mg (0.10 mmol) of compound 70 are quaternized with methyl iodide in analogy to the synthesis of compound Ex. C, and 62 mg of ammonium salt example J are obtained as a colorless solid with the molecular weight of 566.24 (C₃₁H₃₄F₂N₃O₅); MS (ESI): 566.48 (M⁺).

### 3-{3-Benzyloxy-4-[3-[3-(tert-butyldimethylsilanyloxy)-3-(4-fluorophenyl)propyl]-1-(4-fluorophenyl)-4-oxoazetidin-2-yl]phenoxy}propyl ammonium sulfate 44

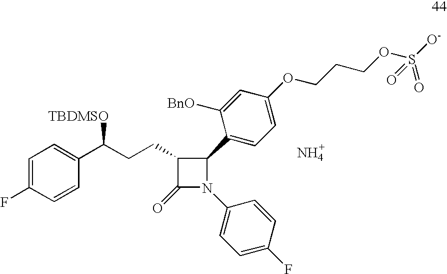

1.21 g (1.9 mmol) of phenol 37 are dissolved in 30 ml of DMF. Addition of 3.0 g (21 mmol) of 1,3-propanediol cyclic sulfate (Aldrich) and 4.5 g of K₂CO₃ is followed by stirring at room temperature for 2 hours. The reaction solution is made slightly acidic with 2 N aqueous HCl and then extracted with ethyl acetate. The organic phase is washed twice with NaCl solution, then basified with methylene chloride/methanol/conc. ammonia (30/5/1) and concentrated. The residue is purified by flash chromatography (methylene chloride/methanol/conc. ammonia 100/7/1 then 30/5/1 then 30110/3), and 1.08 g of ammonium salt 44 are obtained.

### 3-{4-[3-[3-(tert-Butyldimethylsilanyloxy)-3-(4-fluorophenyl)propyl]-1-(4-fluorophenyl)-4-oxoazetidin-2-yl]-3-hydroxyphenoxy}propyl ammonium sulfate 45

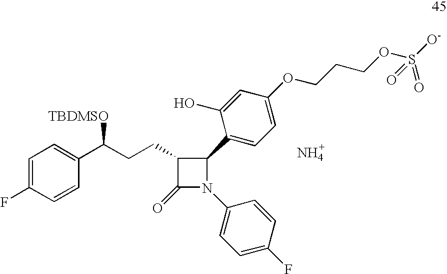

1.07 g (1.39 mmol) 44 are hydrogenated in 20 ml of methanol with 200 mg of Pd/C (10% Pd) under 5 bar of hydrogen for 2 hours, and 979 mg of crude product 45 are obtained.

### 3-(4-{1-(4-Fluorophenyl)-3-[3-(4-fluorophenyl)-3-hydroxypropyl]-4-oxoazetidin-2-yl}-3-hydroxyphenoxy)propyl ammonium sulfate, Example K

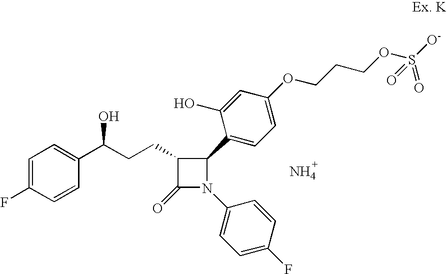

1.5 g of crude product 45 are left to stand in a mixture of 40 ml of THF and 10 ml of 2 N aqueous HCl for 18 hours. The solution is diluted with 50 ml of methylene chloride/methanol/conc. ammonia (30/10/3) (until the solution is basic) and concentrated. The residue is suspended in 30/5/1 and filtered through a little silica gel, washed with 30/10/3 and concentrated, and the resulting residue is separated by flash chromatography (methylene chloride/methanol/conc. ammonia 30/5/1 then 30/10/3 then 30/15/5). 1.03 g of ammonium salt Ex. K are obtained with the molecular weight of 563.14 (C₂₇H₂₇F₂NO₈S); MS (ESI⁺): 1127.14 (2M+H⁺).

### (4-Iodobutoxymethyl)benzene 46

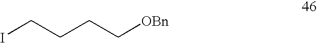

10 g (55.5 mmol) of 4-benzyloxy-1-butanol (Aldrich) are dissolved in 200 ml of toluene. This is followed by addition successively of 6 g (88 mmol) of imidazole, 17 g (64 mmol) of triphenylphosphine and then of 17 g (67 mmol) of iodine while stirring vigorously. After stirring for 2 hours, 200 ml of saturated sodium bicarbonate solution are added, and iodine is added until the organic phase remains dark. The excess iodine is reduced with 10% strength thiosulfate solution, and the organic phase is separated off, washed once with NaCl solution, filtered through silica gel and then concentrated. The precipitated triphenylphosphine oxide is separated off, and the mother liquor is concentrated. 15.8 g of crude product 46 are obtained.

### 4-[2-Benzyloxy-4-(4-benzyloxybutoxy)phenyl]-3-[3-(tert-butyldimethylsilanyloxy)-3-(4-fluorophenyl)propyl]-1-(4-fluorophenyl)azetidin-2-one 47

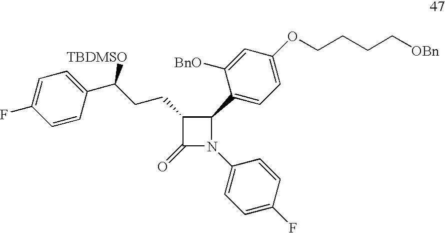

0.5 g (0.79 mmol) of phenol 37 are alkylated with 700 mg (2.4 mmol) of iodide 46 and 650 mg of K₂CO₃ in 15 ml of DMF in analogy to the synthesis of compound 44, and 300 mg of 47 are obtained as an amorphous solid.

### 3-[3-(tert-Butyldimethylsilanyloxy)-3-(4-fluorophenyl)propyl]-1-(4-fluorophenyl)-4-[2-hydroxy-4-(4-hydroxybutoxy)phenyl]azetidin-2-one 48

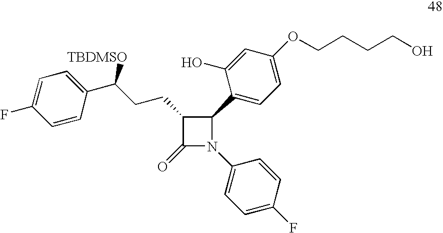

300 mg (0.38 mmol) of 47 are hydrogenated in 3 ml of methanol with 60 mg of Pd/C (10% Pd) under 5 bar of hydrogen for 18 hours. Flash chromatography results in 155 mg of alcohol 48.

### 4-{4-[3-[3-(tert-Butyldimethylsilanyloxy)-3-(4-fluorophenyl)propyl]-1-(4-fluorophenyl)-4-oxoazetidin-2-yl]-3-hydroxyphenoxy}butyl ammonium sulfate 49

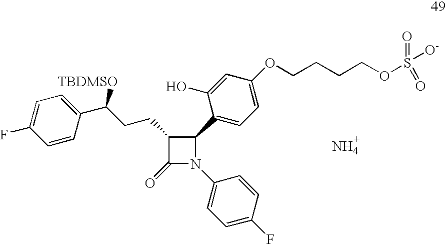

150 mg (0.25 mmol) of 48 are dissolved in 2 ml of pyridine. Addition of 170 mg of trimethylamine-sulfur trioxide complex is followed by stirring at room temperature for 1 hour. The mixture is then diluted with 10 ml of methanol and 10 ml of toluene and concentrated. The residue is passed through a flash chromatography column (methylene chloride/methanol/conc. ammonia 30/5/1 then 30/10/3), and 150 mg of sulfate 49 are obtained.

### 4-(4-{1-(4-Fluorophenyl)-3-[3-(4-fluorophenyl)-3-hydroxypropyl]-4-oxoazetidin-2-yl}-3-hydroxyphenoxy)butyl ammonium sulfate, Example L

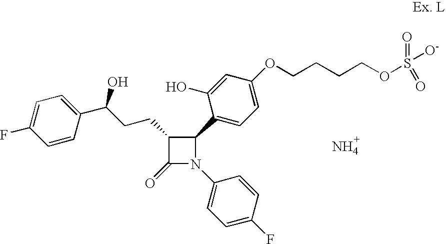

150 mg (0.22 mmol) of 49 are TBDMS-deprotected in analogy to the synthesis of compound example K, and 69 mg of sulfate example L are obtained with the molecular weight of 577.19 (C₂₈H₂₉F₂NO₈S); MS (ESI⁺): 578.26 (M+H⁺).

### 4-[2-Benzyloxy-4-(4-bromobut-2-enyloxy)phenyl]-3-[3-(tert-butyldimethylsilanyloxy)-3-(4-fluorophenyl)propyl]-1-(4-fluorophenyl)azetidin-2-one 50

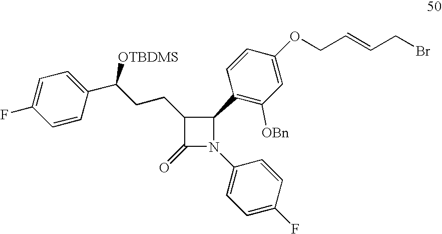

200 mg (0.32 mmol) of phenol 37 are alkylated with 600 mg (2.8 mmol) of 1,4-dibromo-2-butene and 600 mg of K₂CO₃ in 5 ml of DMF, and 220 mg of bromide 50 are obtained.

### 4-[2-Benzyloxy-4-(4-imidazol-1-ylbut-2-enyloxy)phenyl]-3-[3-(tert-butyldimethylsilanyloxy)-3-(4-fluorophenyl)propyl]-1-(4-fluorophenyl)azetidin-2-one 51

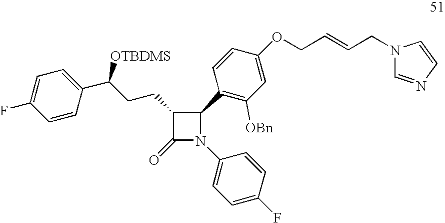

210 mg (0.28 mmol) of 50 are dissolved in 5 ml of toluene and, after addition of 400 mg of imidazole, stirred at 60° C. for one hour. The reaction solution is concentrated and the residue is purified by flash chromatography (methylene chloride/methanol/conc. ammonia 100/7/1 then 30/5/1). 155 mg of product 51 are obtained.

### 1-(4-Fluorophenyl)-3-[3-(4-fluorophenyl)-3-hydroxypropyl]-4-[2-hydroxy-4-(4-imidazol-1-ylbutoxy)phenyl]azetidin-2-one, Compound 71

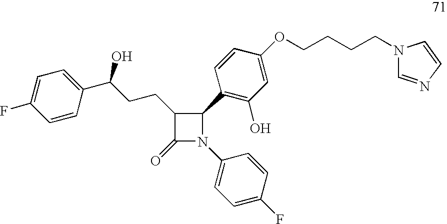

155 mg (0.21 mmol) of 51 are benzyl and silyl-deprotected in analogy to the synthesis of Ex. K, and 75 mg of imidazole derivative 71 are obtained with the molecular weight of 547.61 (C₃₁H₃₁F₂N₃O₄); MS (ESI⁺): 548.23 (M+H⁺).

### 3-[4-(4-{1-(4-Fluorophenyl)-3-[3-(4-fluorophenyl)-3-hydroxypropyl]-4-oxoazetidin-2-yl}-3-hydroxyphenoxy)butyl]-1-methyl-3H-imidazol-1-ium iodide, Example M

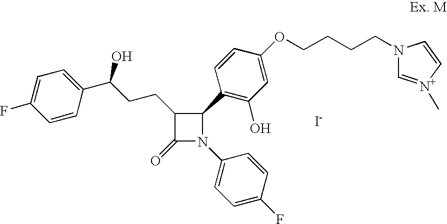

55 mg (0.10 mmol) of 71 are dissolved in 2 ml of toluene. Addition of 2 ml of methyl iodide is followed by boiling under reflux at an oil bath temperature of 80° C. for 3 hours. The precipitated product is filtered off with suction to result in 46 mg of Ex. M as a colorless solid with the molecular weight of 562.25 (C₃₂H₃₄F₂N₃O₄); MS (ESI): 562.11 (M⁺).

### 4-[4-Benzyloxy-2-(2-trimethylsilanylethoxymethoxy)phenyl]-3-[3-(tert-butyldimethylsilanyloxy)-3-(4-fluorophenyl)propyl]-1-(4-fluorophenyl)azetidin-2-one 53

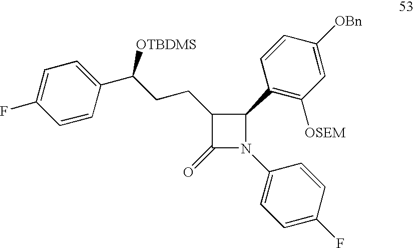

1.0 g (1.59 mmol) of phenol 39 is reacted with 0.8 ml of SEMCI (Aldrich) and 1.2 g of K₂CO₃ in 5 ml of DMF, and 1.3 g of SEM-protected phenol 53 are obtained.

### 3-[3-(tert-Butyldimethylsilanyloxy)-3-(4-fluorophenyl)propyl]-1-(4-fluorophenyl)-4-[4-hydroxy-2-(2-trimethylsilanylethoxymethoxy)phenyl]azetidin-2-one 54

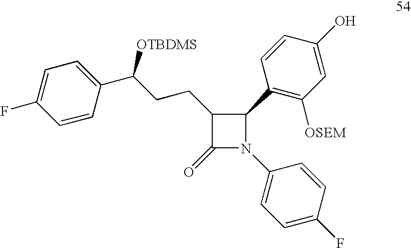

1.1 g (1.4 mmol) of 53 are hydrogenated with Pd/C in analogy to the synthesis of compound 14, and 0.9 g of phenol 54 is obtained.

### 4-[4-(4-Bromomethylbenzyloxy)-2-(2-trimethysilanylethoxymethoxy)phenyl]-3-[3-(tert-butyldimethylsilanyloxy)-3-(4-fluorophenyl)propyl]-1-(4-fluorophenyl)azetidin-2-one 55

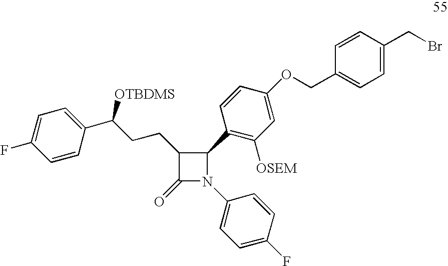

0.9 g (1.3 mmol) of phenol 54 is reacted with 2.5 g (9.5 mmol) of p-xylene dibromide (Aldrich) and 2.5 g of K₂CO₃ in 30 ml of DMF, and 0.6 g of bromide 55 is obtained.

### 4-[4-(4-Bromomethylbenzyloxy)-2-hydroxyphenyl]-1-(4-fluorophenyl)-3-[3-(4-fluorophenyl)-3-hydroxypropyl]azetidin-2-one 56

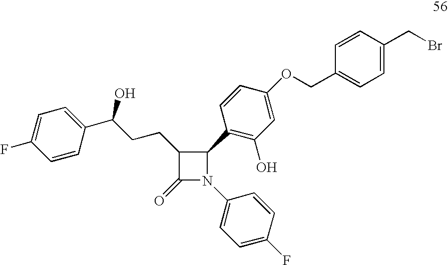

0.6 g (0.7 mmol) of 55 is dissolved in 30 ml of THF. Addition of 6 ml of 2 N aqueous HCl is followed by heating at 60° C. for 7 hours. The reaction solution is diluted with 100 ml of ethyl acetate, the aqueous phase is separated off, and the organic phase is washed twice with NaCO₃ solution, filtered through a little silica gel and concentrated. The residue is separated by flash chromatography (nheptane/ethyl acetate 1:1), and 340 mg of deprotected bromide 56 are obtained.

### 1-[4-(4-{1-(4-Fluorophenyl)-3-[3-(4-fluorophenyl)-3-hydroxypropyl]-4-oxoazetidin-2-yl}-3-hydroxyphenoxymethyl)benzyl]-4-aza-1-azoniabicyclo[2.2.2]octane bromide, Example N

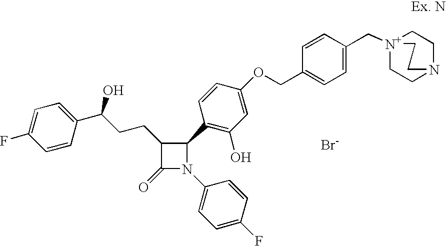

122 mg (0.20 mmol) of 56 are dissolved in 5 ml of toluene and stirred with 150 mg of DABCO at 80° C. for 2 hours. The precipitated product is filtered off with suction and washed with toluene, and 138 mg of trialkylammoniumalkyl salt Ex. N are obtained with the molecular weight of 640.30 (C₃₈H₄₀F₂N₃O₄); MS (ESI): 640.38 (M⁺).

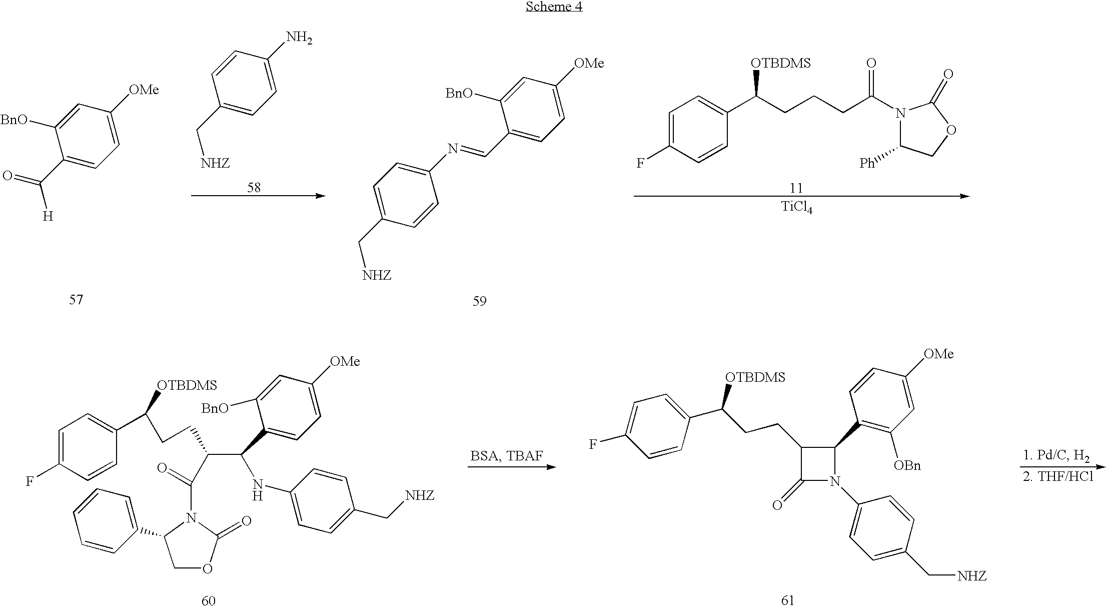

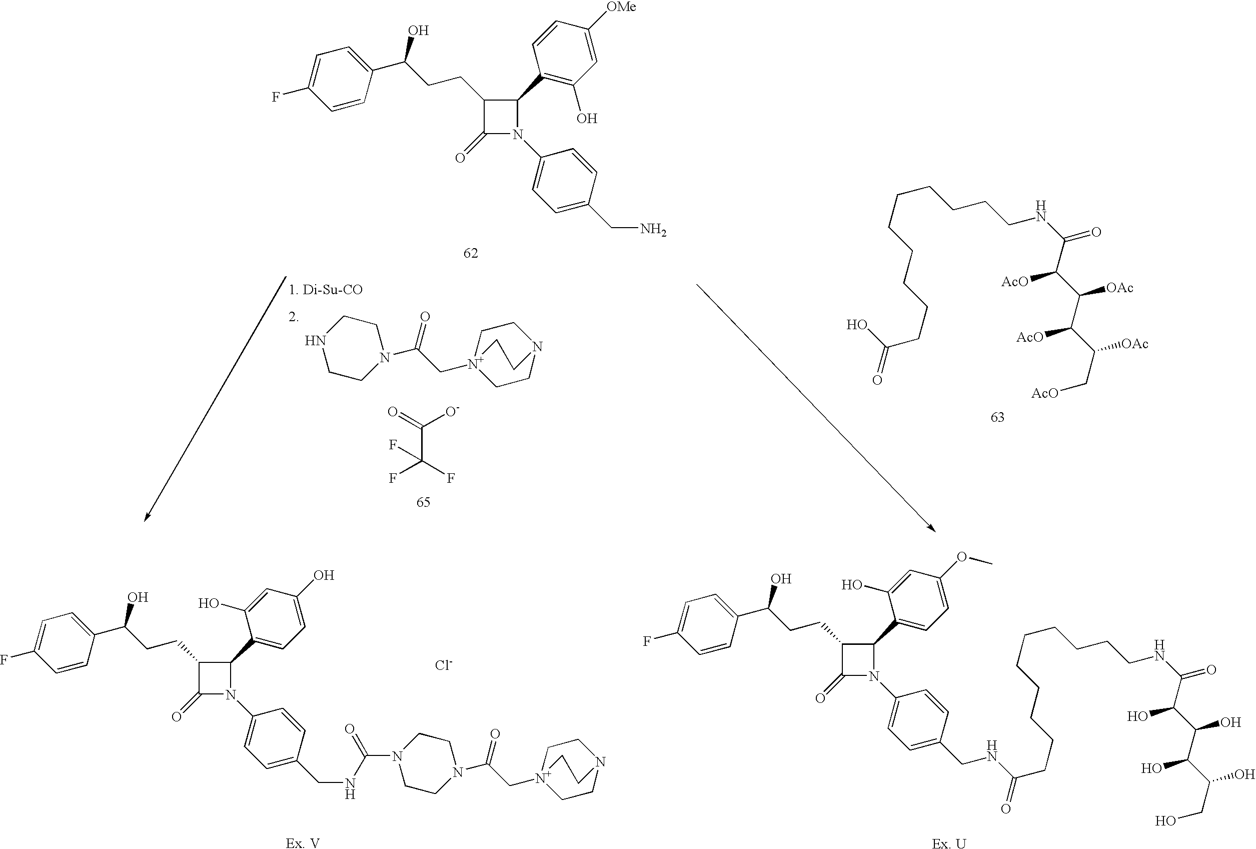

### Benzyl {4-[(2-benzyloxy-4-methoxybenzylidene)amino]benzyl}carbamate 59

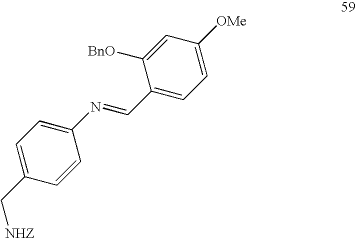

8 g (33 mmol) of aldehyde 57 and 10 g (crude product) of aniline 58 are reacted in analogy to the synthesis of imine 10, and 6.5 g of crystalline product 59 are obtained.

### Benzyl {4-[1-(2-benzyloxy-4-methoxyphenyl)-5-(tert-butyldimethylsilanyloxy)-5-(4-fluorophenyl)-2-(2-oxo-4-phenyloxazolidine-3-carbonyl)pentylamino]benzyl carbamate 60

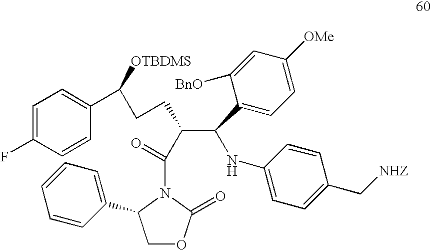

2.5 g (7.0 mmol) of oxazolidinone 11 and 3.0 g (6.2 mmol) of imine 59 are reacted in analogy to the synthesis of compound 12, and 1.78 g of product 60 and 1.5 g of recovered precursor 11 are obtained.

### Benzyl (4-{2-(2-benzyloxy-4-methoxyphenyl)-3-[3-(tert-butyldimethylsilanyloxy)-3-(4-fluorophenyl)propyl]-4-oxoazetidin-1-yl}benzyl)carbamate 61

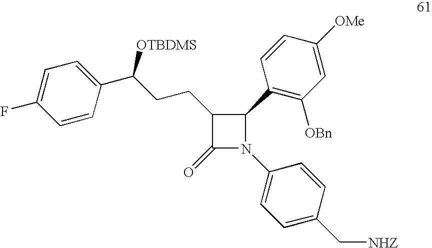

1.78 g (1.9 mmol) of 60 are cyclized in analogy to the synthesis of beta-lactam 13, and 880 mg of product 61 are obtained.

### 1-(4-Aminomethylphenyl)-3-[3-(4-fluorophenyl)-3-hydroxypropyl]-4-(2-hydroxy-4-methoxyphenyl)azetidin-2-one 62

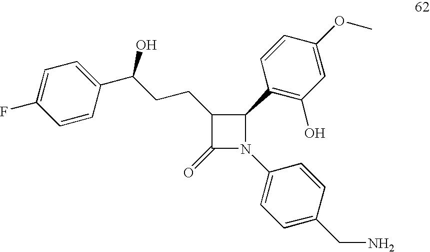

860 mg (1.1 mmol) of 61 are benzyl and silyl-deprotected in analogy to the synthesis of Ex. O, and 300 mg of beta-lactam 62 are obtained as an amorphous solid.

### 11-(2,3,4,5,6-Pentahydroxyhexanoylamino)undecanoic acid 4-[3-[3-(4-fluorophenyl)-3-hydroxypropyl]-2-(2-hydroxy-4-methoxyphenyl)-4-oxoazetidin-1-yl]benzylamide, Example O

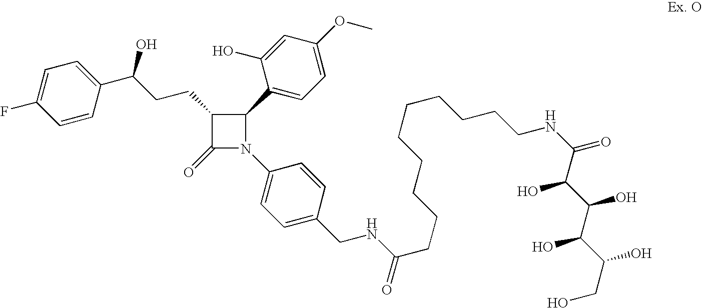

60 mg (0.11 mmol) of benzylamine 62 are reacted with 200 mg of acid 63, 150 mg of EDC and 75 mg of HOBt in 5 ml of DMF in analogy to example G (16 hours at room temperature), and 50 mg of acylated Ex. O are obtained. This intermediate is deprotected with MeOH/NaOMe, and 29 mg of sugar derivative Ex. O are isolated as an amorphous solid with the molecular weight of 811.95 (C₄₃H₅₈FN₃O₁₁); MS (ESI⁺): 812.42 (M+H⁺).

### 2,5-Dioxopyrrolidin-1-yl {4-[3-[3-(4-fluorophenyl)-3-hydroxyproyl]-2-(2-hydroxy-4-methoxyphenyl)-4-oxoazetidin-1-yl]benzyl}carbamate 64

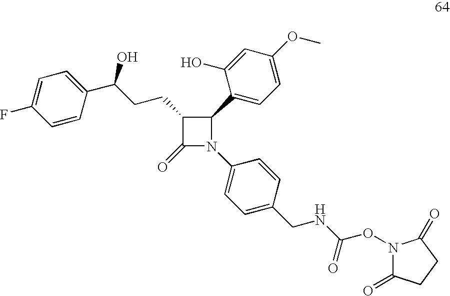

100 mg (0.22 mmol) of 62 are dissolved in 5 ml of acetonitrile and stirred with 100 mg of Di-Su-CO at room temperature for one hour. The residue after concentration of the reaction solution is purified by flash chromatography (n-heptane/ethyl acetate 1:2 then 0:1), and 60 mg of 64 are obtained.

### 1-[2-(4-{4-[3-[3-(4-Fluorophenyl)-3-hydroxypropyl]-2-(2-hydroxy-4-methoxyphenyl)-4-oxoazetidin-1-yl]benzylcarbamoyl}piperazin-1-yl)-2-oxoethyl]-4-aza-1-azoniabicyclo[2.2.2]octane chloride, Example P

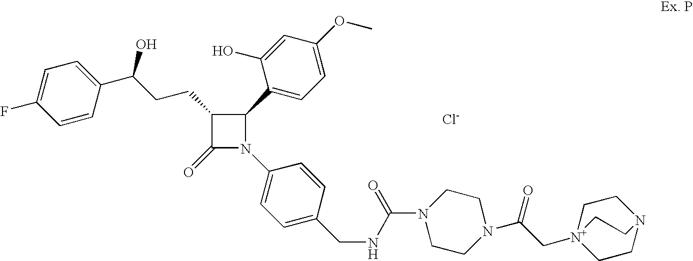

60 mg (0.10 mmol) of 64 are dissolved in 3 ml of acetonitrile. Addition of 40 mg of piperazine derivative 65 and 0.3 ml of 1 N aqueous sodium hydroxide solution is followed by stirring at room temperature for 4 hours. The reaction solution is concentrated to 1 ml, diluted with 1 ml of DMF and purified by preparative HPLC. 23 mg of ammonium salt Ex. P are obtained with the molecular weight of 715.36 (C₃₉H₄₈FN₆O₆); MS (ESI): 715.34 (M⁺).

The compounds of the invention of the formula I were tested for their effect by the method described below:

### Influence on Cholesterol Absorption+³H-Taurocholic Acid Excretion on the Basis of the Fecal Excretion in Mice, Rats or Hamsters

NMRI mice, Wistar rats, or golden Syrian hamsters (in groups of n=4-6) are kept with a standard diet (Altromin, Lage (Lippe)) in metabolism cages. The animals are fasted from the afternoon before administration of the radioactive tracer (¹⁴C-cholesterol) and adapted to wire grids.

In addition, the animals are labeled with ³H-TCA (taurocholic acid) s.c. 24 hours before the oral administration of the test meal (¹⁴C-cholesterol in Intralipid® 20, Pharmacia-Upjohn) (e.g. 1 μCi/mouse to 5 μCi/rat).

Cholesterol absorption test: 0.25 ml/mouse Intralipid® 20 (Pharmacia-Upjohn) (spiking with 0.25 μCi ¹⁴C-cholesterol in 0.1 mg of cholesterol) is administered orally by gavage.

Test substances are made up separately in 0.5%/(methylcellulose (Sigma)/5% Solutol (BASF, Ludwigshafen) or a suitable vehicle.

The volume for administration of the test substance is 0.5 ml/mouse. The test substance is administered orally immediately before the test meal (Intralipid with ¹⁴C-cholesterol label) (cholesterol absorption test).

Feces are collected for 24 h: the fecal elimination of ¹⁴C-cholesterol and ³H taurocholic acid (TCA) is determined after 24 h.

The livers are removed, homogenized and combusted in aliquots in an oximate (model 307, Packard) to determine the amount of ¹⁴C-cholesterol taken up/absorbed.

### Evaluation:

### Feces Samples:

The total weight is determined, and the material is made up to a defined volume with water and then homogenized, and an aliquot is dried and combusted in an oximate (model 307, Packard for combustion of radiolabeled samples). The amount of radioactive ³H- H₂O and ¹⁴C- CO₂ is extrapolated to the excreted amount of ³H-taurocholic acid or ¹⁴C-cholesterol (dual isotope technique). The ED₂₀₀ values are interpolated as dose from a dose-effect plot as the doses which double the excretion of TCA or cholesterol relative to a control group treated at the same time.

### Liver Samples:

The amount of ¹⁴C-cholesterol taken up in the liver is evaluated in relation to a control group (vehicle-treated) as a function of the dose of the substance administered. The ED₅₀ values are interpolated from a dose-effect plot as the dose which halves (50%) the uptake of ¹⁴C-cholesterol in the liver relative to a control group.

The following ED₅₀ values (liver value; mouse) demonstrate the activity of the compounds of the invention of the formula I

| Example No. | ED₅₀ (liver) [mg/mouse] |
|---|---|
| O | <0.01 |
| M | <0.1 |
| A | 0.01 |
| P | 0.3 |
| B | <0.1 |
| I | 0.01 |
| H | 0.01 |
| F | 0.3 |
| E | <0.01 |
| C | 0.1 |
| K | <0.01 |
| N | 0.01 |

It is evident from the table that the compounds of the formula I very efficiently inhibit uptake of cholesterol from the gastrointestinal tract.

### Hamster Model:

Syrian hamsters (in groups of n=5-9) receive a standard diet (ssniff, Soest Germany) supplemented with 0.1% cholesterol.

The test substances are made up in 0.5%/(methylcellulose (Sigma)/5% Solutol (BASF, Ludwigshafen) or another suitable vehicle, and at least three dosages are administered once a day by gavage on 12 consecutive days.

On day 12, the animals are exsanguinated from the aorta under deep anesthesia. The serum is analyzed for total cholesterol, LDL-cholesterol, HDL-cholesterol and triglycerides using standard kits from Roche in accordance with the guidelines of the German Society for Clinical Chemistry.

The ED₅₀ values for LDL cholesterol reduction compared with placebo-treated control animals were calculated using a standard logistic model for the dose-effect plot.

Data from Hamster Experiments:

The following ED₅₀ values (serum LDL cholesterol value; hamster, [mg/kg]) demonstrate the activity of the compounds of the invention of the formula I:

| Example No. | ED₅₀ |
|---|---|
| A | 0.2 |
| I | <0.1 |
| E | 0.1 |

### Liver Exposure:

The liver exposure of the compounds of the invention compared with the corresponding compounds without a hydroxyl function in the 2″ position was investigated in vivo in male Wistar rats. A rat anesthetized with ketamine/midazolam (ketamine 80 mg/kg i.p.+midazolam 5 mg/kg i.p.) undergoes laparotomy in the Linea alba, and then the product is administered intraduodenally. During this it is attempted to prevent direct reflux of the product into the stomach. The animals remain under anesthesia throughout the experiment. At the end of the experiment, after 2 h, the liver is dissected out for determination of the substance. The substance levels in the liver homogenates are determined by LC-MS/MS. For this purpose, initially the proteins are precipitated by adding acetonitrile in the presence of an internal standard. Part of the supernatant is mixed with a suitable buffer, and an aliquot of the mixture is injected. The measurement is evaluated via the peak areas of analyte and internal standard.

### Results:

Cₘₐₓ values in the liver (L) after oral administration of 10 mg/kg of body weight

| “H compounds” | “2″-OH compounds” |
|---|---|
| 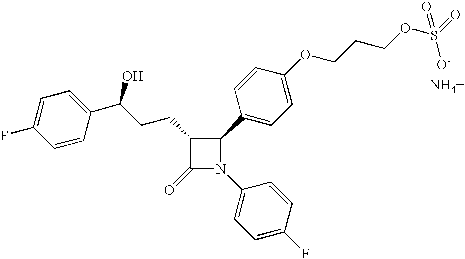 | 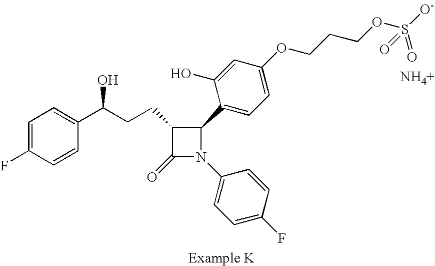 |
| L = 24.3 μg/mL | L = 0.30 μg/mL |
| 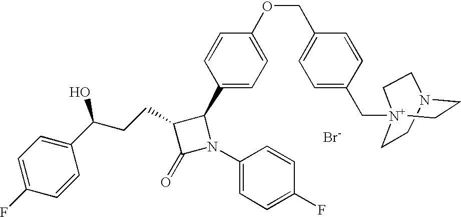 | 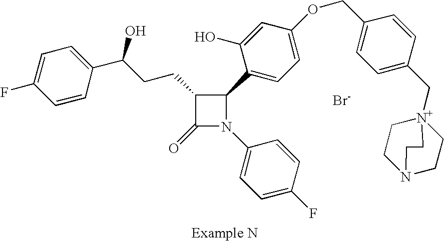 |
| L = 0.20 μg/mL | L = 0.08 μg/mL |

It is evident from these data that the compounds of the invention display distinctly lower liver levels and thus reduce the stress on the liver.

What is claimed is:

1. A compound of formula I 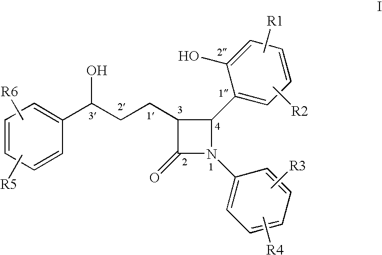
wherein:
R1, R2, R3, R4, R5 and R6 are independently selected from the group consisting of:
(C₁-C₃₀)-alkylene-(LAG)ₙ, where n may be 1-5, and where one or more carbon atoms of the alkylene group may be replaced by —S(O)ₙ—, with n=0-2, —O—, —(C═O)—, —(C═S)—, —CH═CH—, —C≡C—, —N((C₁-C₆)-alkyl)-, —N(phenyl)-, —N((C₁-C₆)-alkylphenyl)-, —N(CO—(CH₂)₁₋₁₀—COOH)—, —N(CO—(C₁-C₈)-alkyl)-, —N(CO—(C₃-C₈)-cycloalkyl), N(CO—(CH₂)₀₋₁₀-aryl), —N(CO—(CH₂)₀₋₁₀-heteroaryl), —NH— or by aryl or heteroaryl groups which are substituted up to three times by R7, or by (C₃-C₁₀)-cycloalkyl or heterocycloalkyl groups which are substituted up to four times by R7;
(C₁-C₃₀)-alkylene-(LAG)ₙ, where n may be 1-5, and where one or more C atoms of the alkylene group may be replaced by —S(O)ₙ—, with n=0-2, —O—, —(C═O)—, —(C═S)—, —CH═CH—, —C═C—, —N((C₁-C₆)-alkyl)-, —N(phenyl)-, —N((C₁-C₆)-alkylphenyl)-, —N(CO—(CH₂)₁₋₁₀—COOH)—, —N(CO—(C₁-C₈)-alkyl)-, —N(CO—(C₃-C₈)-cycloalkyl), N(CO—(CH₂)₀₋₁₀-aryl), —N(CO—(CH₂)₀₋₁₀-heteroaryl), —NH— or by aryl or heteroaryl groups which may be substituted up to three times by R7, or by (C₃-C₁₀)-cycloalkyl or hetero-cycloalkyl groups which may be substituted up to four times by R7;
H, F, Cl, Br, I, CF₃, NO₂, N₃, CN, COOH, COO(C₁-C₆)alkyl, CONH₂, CONH(C₁-C₆)alkyl, CON[(C₁-C₆)alkyl]₂, (C₁-C₆)-alkyl, (C₂-C₆)-alkenyl, (C₂-C₆)-alkynyl, O—(C₁-C₆)-alkyl, where one, more than one, or all hydrogen(s) in the alkyl groups may be replaced by fluorine;
C(═NH)(NH₂), PO₃H₂, SO₃H, SO₂—NH₂, SO₂NH(C₁-C₆)-alkyl, SO₂N[(C₁-C₆)-alkyl]₂, S—(C₁-C₆)-alkyl, S—(CH₂)ₙ-phenyl, SO—(C₁-C₆)-alkyl, SO—(CH₂)ₙ-phenyl, SO₂—(C₁-C₆)-alkyl, SO₂—(CH₂)ₙ-phenyl, where n may be 0-6, and the phenyl group may be substituted up to twice by F, Cl, Br, OH, CF₃, NO₂, CN, OCF₃, O—(C₁-C₆)-alkyl, (C₁-C₆)-alkyl, NH₂;
NH₂, NH—(C₁-C₆)-alkyl, N((C₁-C₆)-alkyl)₂, NH(C₁-C₇)-acyl, phenyl, O—(CH₂)ₙ-phenyl, where n may be 0-6, where the phenyl ring may be substituted one to three times by F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl, and CONH₂;
R7 is selected from the group consisting of F, Cl, Br, I, CF₃, NO₂, N₃, CN, COOH, COO(C₁-C₆)alkyl, CONH₂, CONH(C₁-C₆)alkyl, CON[(C₁-C₆)alkyl]₂, (C₁-C₆)-alkyl, (C₂-C₆)-alkenyl, (C₂-C₆)-alkynyl, O—(C₁-C₆)-alkyl, where one, more than one, or all hydrogen(s) in the alkyl groups may be replaced by fluorine; C(═NH)(NH₂), PO₃H₂, SO₃H, SO₂—NH₂, SO₂NH(C₁-C₆)-alkyl, SO₂N[(C₁-C₆)-alkyl]₂, S—(C₁-C₆)-alkyl, S—(CH₂)ₙ-phenyl, SO—(C₁-C₆)-alkyl, SO—(CH₂)ₙ-phenyl, SO₂—(C₁-C₆)-alkyl, SO₂—(CH₂)ₙ-phenyl, where n may be 0-6, and the phenyl group may be substituted up to twice by F, Cl, Br, OH, CF₃, NO₂, CN, OCF₃, O—(C₁-C₆)-alkyl, (C₁-C₆)-alkyl, NH₂;
NH₂, NH—(C₁-C₆)-alkyl, N((C₁-C₆)-alkyl)₂, NH(C₁-C₇)-acyl, aryl, O—(CH₂)ₙ-aryl, where n may be 0-6, where the aryl ring may be substituted one to three times by F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl, and CONH₂; and,
LAG represents a C₄-C₁₀-cycloaliphatic group substituted by 2 to 9 hydroxy functions, or a C₂-C₁₀-aliphatic group substituted by 2 to 10 hydroxy functions, where in each case one or more hydroxy functions may be replaced by an —NHR8 group; an amino acid residue, an oligopeptide residue consisting of 2 to 9 amino acids; an acyclic, mono- or bicyclic tri-alkylammonium group, acyclic, mono- or bicyclic tri-alkylammoniumalkyl group, where up to three carbon atoms may be replaced by N, O or S(O), with n=0-2; N-alkylated heteroaromatics such as, for example, imidazolium or pyridinium; —O—(SO₂)—OH; —(CH₂)₀₋₁₀—SO₃H; —(CH₂)₀₋₁₀—P(O)(OH)₂, —(CH₂)₀₋₁₀—O—P(O)(OH)₂, —(CH₂)₀₋₁₀—C(═NH)(NH₂); —(CH₂)₀₋₁₀—C(═NH)(NHOH); —NR8-C(═NR9)(NR10R11); where n is 1-5, and
R8, R9, R10 and R11 are independently selected from the group consisting of H, (C₁-C₆)-alkyl, phenyl, (C₁-C₆)-alkyl-phenyl, (C₃-C₈)-cycloalkyl, —C(O)—(C₁-C₆)-alkyl, —C(O)—(C₃-C₈)-cycloalkyl; and where at least one of the R1 to R6 groups must be a (C₁-C₃₀)-alkylene-(LAG)ₙ, where n may be 1-5, and where one or more carbon atoms of the alkylene group may be replaced by —S(O)ₙ—, with n=0-2, —O—, —(C═O)—, —(C═S)—, —CH═CH—, —C≡C—, —N((C₁-C₆)-alkyl)-, —N(phenyl)-, —N((C₁-C₆)-alkylphenyl)-, —N(CO—(CH₂)₁₋₁₀—COOH)—, —N(CO—(C₁-C₈)-alkyl)-, —N(CO—(C₃-C₈)-cycloalkyl), N(CO—(CH₂)₀₋₁₀-aryl), —N(CO—(CH₂)₀₋₁₀-heteroaryl), —NH— or by aryl or heteroaryl groups which are substituted up to three times by R7, or by (C₃-C₁₀)-cycloalkyl or heterocycloalkyl groups which may optionally are substituted up to four times by R7;
and the pharmaceutically acceptable salts thereof.

2. The compound of formula I as recited in claim 1, wherein
R1, R2, R3, R4, R5 and R6 are independently selected from the group consisting of (C₁-C₂₀)-alkylene-(LAG), where one or more carbon atoms of the alkylene group may be replaced by —O—, —(C═O)—, —N((C₁-C₆)-alkyl)-, —N(CO—(CH₂)₁₋₁₀—COOH)— or —NH— or by aryl or heteroaryl groups which are substituted up to three times by R7 or by (C₃-C₁₀)-cycloalkyl or heterocycloalkyl groups which may be substituted up to four times by R7; H, F, Cl, Br, I, CF₃, NO₂, N₃, CN, COOH, COO(C₁-C₆)alkyl, CONH₂, CONH(C₁-C₆)alkyl, CON[(C₁-C₆)alkyl]₂, (C₁-C₆)-alkyl, (C₂-C₆)-alkenyl, (C₂-C₆)-alkynyl, O—(C₁-C₆)-alkyl, where at least one hydrogen(s) in the alkyl groups may be replaced by fluorine;
C(═NH)(NH₂), PO₃H₂, SO₃H, SO₂—NH₂, SO₂NH(C₁-C₆)-alkyl, SO₂N[(C₁-C₆)-alkyl]₂, S—(C₁-C₆)-alkyl, S—(CH₂)ₙ-phenyl, SO—(C₁-C₆)-alkyl, SO—(CH₂)ₙ-phenyl, SO₂—(C₁-C₆)-alkyl, SO₂—(CH₂)ₙ-phenyl, where n may be 0-6, and the phenyl group may be substituted up to twice by F, Cl, Br, OH, CF₃, NO₂, CN, OCF₃, O—(C₁-C₆)-alkyl, (C₁-C₆)-alkyl, NH₂; NH₂, NH—(C₁-C₆)-alkyl, N((C₁-C₆)-alkyl)₂, NH(C₁-C₇)-acyl, phenyl, O—(CH₂)ₙ-phenyl, where n may be 0-6, where the phenyl ring may be substituted one to three times by F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl, CONH₂;
R7 is selected from the group consisting of F, Cl, Br, I, CF₃, NO₂, N₃, CN, COOH, COO(C₁-C₆)alkyl, CONH₂, CONH(C₁-C₆)alkyl, CON[(C₁-C₆)alkyl]₂, (C₁-C₆)-alkyl, (C₂-C₆)-alkenyl, (C₂-C₆)-alkynyl, O—(C₁-C₆)-alkyl, where one, more than one, or all hydrogen(s) in the alkyl groups may be replaced by fluorine; C(═NH)(NH₂), PO₃H₂, SO₃H, SO₂—NH₂, SO₂NH(C₁-C₆)-alkyl, SO₂N[(C₁-C₆)-alkyl]₂, S—(C₁-C₆)-alkyl, S—(CH₂)ₙ-phenyl, SO—(C₁-C₆)-alkyl, SO—(CH₂)ₙ-phenyl, SO₂—(C₁-C₆)-alkyl, SO₂—(CH₂)ₙ-phenyl, where n may be 0-6, and the phenyl group may be substituted up to twice by F, Cl, Br, OH, CF₃, NO₂, CN, OCF₃, O—(C₁-C₆)-alkyl, (C₁-C₆)-alkyl, NH₂;
NH₂, NH—(C₁-C₆)-alkyl, N((C₁-C₆)-alkyl)₂, NH(C₁-C₇)-acyl, aryl, O—(CH₂)ₙ-aryl, where n may be 0-6, where the aryl ring may be substituted one to three times by F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl, CONH₂;
LAG is selected from the group consisting of a C₄-C₁₀-cycloaliphatic group substituted by 2 to 9 hydroxy functions, or C₂-C₁₀-aliphatic groups substituted by 2 to 10 hydroxy functions, where in each case one or more hydroxy functions may be replaced by an —NHR8 group; amino acid residue, oligopeptide residue consisting of 2 to 9 amino acids; acyclic, mono- or bicyclic trialkylammonium group, acyclic, mono- or bicyclic trialkylammoniumalkyl group, where up to three carbon atoms may be replaced by N, O or S(O), with n=0-2; N-alkylated heteroaromatics such as, for example, imidazolium or pyridinium;
—O—(SO₂)—OH; —(CH₂)₀₋₁₀—SO₃H; —(CH₂)₀₋₁₀—P(O)(OH)₂, —(CH₂)₀₋₁₀—O—P(O)(OH)₂, —(CH₂)₀₋₁₀—C(═NH)(NH₂); —(CH₂)₀₋₁₀—C(═NH)(NHOH); —NR8-C(═NR9)(NR10R11); where n is 1-5, and R8, R9, R10 and R11 may independently of one another be H, (C₁-C₆)-alkyl, phenyl, (C₁-C₆)-alkyl-phenyl, (C₃-C₈)-cycloalkyl), —C(O)—(C₁-C₆)-alkyl, —C(O)—(C₃-C₈)-cycloalkyl;
wherein at least one of the R1 to R6 groups is a (C₁-C₂₀)-alkylene-(LAG)ₙ, where one or more C atoms of the alkylene group may be replaced by —O—, —(C═O)—, —N((C₁-C₆)-alkyl)-, —N(CO—(CH₂)₁₋₁₀—COOH)—, —NH— or by aryl or heteroaryl groups which are substituted up to three times by R7, or by (C₃-C₁₀)-cycloalkyl or heterocycloalkyl groups which are substituted up to four times by R7; and the pharmaceutically acceptable salts thereof.

3. The compound of formula I as recited in claim 2, wherein:
R1 and R3 are independently selected from the group consisting of H or (C₁-C₁₂)-alkylene-(LAG), where one or more C atoms of the alkylene group may be replaced by —O—, —(C═O)—, —N(CH₃)—, or —NH— or by aryl or heteroaryl groups which are substituted up to three times by R7 or by (C₃-C₁₀)-cycloalkyl or heterocycloalkyl groups which are substituted up to four times by R7, with always one of the groups R1 and R3 always having the meaning of H and the other one having the meaning of (C₁-C₁₂)-alkylene-(LAG);
R2, R4, R5 and R6 are selected from the group consisting of H;
R7 is selected from the group consisting of F, Cl, Br, I, CF₃, NO₂, N₃, CN, COOH, COO(C₁-C₆)alkyl, CONH₂, CONH(C₁-C₆)alkyl, CON[(C₁-C₆)alkyl]₂, (C₁-C₆)-alkyl, (C₂-C₆)-alkenyl, (C₂-C₆)-alkynyl, O—(C₁-C₆)-alkyl, where one, more than one, or all hydrogen(s) in the alkyl groups may be replaced by fluorine;
C(═NH)(NH₂), PO₃H₂, SO₃H, SO₂—NH₂, SO₂NH(C₁-C₆)-alkyl, SO₂N[(C₁-C₆)-alkyl]₂, S—(C₁-C₆)-alkyl, S—(CH₂)ₙ-phenyl, SO—(C₁-C₆)-alkyl, SO—(CH₂)ₙ-phenyl, SO₂—(C₁-C₆)-alkyl, SO₂—(CH₂)ₙ-phenyl, where n may be 0-6, and the phenyl group may be substituted up to twice by F, Cl, Br, OH, CF₃, NO₂, CN, OCF₃, O—(C₁-C₆)-alkyl, (C₁-C₆)-alkyl, NH₂;
NH₂, NH—(C₁-C₆)-alkyl, N((C₁-C₆)-alkyl)₂, NH(C₁-C₇)-acyl, aryl, O—(CH₂)ₙ-aryl, where n may be 0-6, where the aryl ring may be substituted one to three times by F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl, CONH₂;
LAG is selected from the group consisting of C₄-C₁₀-cycloaliphatic group substituted by 2 to 9 hydroxy functions, or C₂-C₁₀-aliphatic groups substituted by 2 to 10 hydroxy functions, where in each case one or more hydroxy functions may be replaced by an —NHR8 group;
amino acid residue, oligopeptide residue consisting of 2 to 9 amino acids; acyclic, mono- or bicyclic trialkylammonium group, acyclic, mono- or bicyclic trialkylammoniumalkyl group, where up to three carbon atoms may be replaced by N, O or S(O), with n=0-2; N-alkylated heteroaromatics such as, for example, imidazolium or pyridinium;
—O—(SO₂)—OH; —(CH₂)₀₋₁₀—SO₃H; —(CH₂)₀₋₁₀—P(O)(OH)₂, —(CH₂)₀₋₁₀—O—P(O)(OH)₂, —(CH₂)₀₋₁₀—C(═NH)(NH₂); —(CH₂)₀₋₁₀—C(═NH)(NHOH); —NR8-C(═NR9)(NR10R11); where n is 1-5, and R8, R9, R10 and R11 may independently of one another be H, (C₁-C₆)-alkyl, phenyl, (C₁-C₆)-alkyl-phenyl, (C₃-C₈)-cycloalkyl, —C(O)—(C₁-C₆)-alkyl, —C(O)—(C₃-C₈)-cycloalkyl; and the pharmaceutically acceptable salts thereof.

4. The compound of formula I as recited in claim 3, wherein
LAG is selected from the group consisting of —(CH₂)₀₋₁₀—O—(SO₂)—OH, —(CH₂)₀₋₁₀—SO₃H or a mono- or bicyclic tri-alkylammonium group in which one to three carbon atoms may be replaced by N, O or S(O)ₙ with n=0-2; and the pharmaceutically acceptable salts thereof.

5. A pharmaceutical composition comprising the compound as defined by formula I in claim 1.

6. The pharmaceutical composition comprising the compound as defined by formula I in claim 4.

7. The pharmaceutical composition comprising the compound as defined by formula I in claim 4 further comprising at least one additional active pharmaceutical compound in a pharmaceutically acceptable carrier.

8. The pharmaceutical composition of claim 7 wherein the additional active pharmaceutical compound is selected from the group consisting of one or more anti-diabetics, hypoglycemic active ingredients, HMGCoA reductase inhibitors, cholesterol absorption inhibitors, PPAR gamma agonists, PPAR alpha agonists, PPAR alpha/gamma agonists, PPAR delta agonists, fibrates, MTP inhibitors, bile acid absorption inhibitors, MTP inhibitors, CETP inhibitors, polymeric bile acid adsorbents, LDL receptor inducers, ACAT inhibitors, antioxidants, lipoprotein lipase inhibitors, ATP-citrate lyase inhibitors, squalene synthetase inhibitors, lipoprotein (a) antagonists, HM74A receptor agonists, lipase inhibitors, insulins, sulfonylureas, biguanides, meglitinides, active ingredients which act on the beta cells, glycogen phosphorylase inhibitors, glucagon receptor antagonists, activators of glucokinase, inhibitors of gluconeogenesis, inhibitors of fructose-1,6-bisphosphatase, modulators of glucose transporter 4, inhibitors of glutamine-fructose-6-phosphate amidotransferase, inhibitors of dipeptidylpeptidase IV, inhibitors of 11-beta-hydroxysteroid dehydrogenase 1, inhibitors of protein tyrosine phosphatase 1B, modulators of the sodium-dependent glucose transporter 1 or 2, GPR40 modulators, inhibitors of hormone-sensitive lipase, inhibitors of acetyl-CoA carboxylase, inhibitors of phosphoenolpyruvate carboxykinase, inhibitors of glycogen synthase kinase-3 beta, inhibitors of protein kinase C beta, endothelin A receptor antagonists, inhibitors of I kappaB kinase, modulators of the glucocorticoid receptor, CART agonists, NPY agonists, MC4 agonists, orexin agonists, H3 agonists, TNF agonists, CRF agonists, CRF BP antagonists, urocortin agonists, β3 agonists, CB1 receptor antagonists, MSH (melanocyte-stimulating hormone) agonists, CCK agonists, serotonin reuptake inhibitors, mixed serotoninergic and noradrenergic compounds, 5HT agonists, bombesin agonists, galanin antagonists, growth hormones, growth hormone-releasing compounds, TRH agonists, uncoupling protein 2 or 3 modulators, leptin agonists, DA agonists (bromocriptine, Doprexin), lipase/amylase inhibitors, peroxisome proliferator activated receptor (PPAR) modulators, RXR modulators or TR-β agonists or amphetamines.

9. A method for the treatment of abnormal blood glucose levels comprising the administration of one or more of the compounds as defined by formula I in claim 1 to a patient in need thereof.

10. A method for the treatment of abnormal blood glucose levels comprising the administration of one or more of the compounds recited in claim 4 to a patient in need thereof.

11. A method for the treatment of type II diabetes comprising the administration of one or more of the compounds recited in claim 1 to a patient in need thereof.

12. A method for the treatment of type II diabetes comprising the administration of one or more of the compounds recited in claim 4 to a patient in need thereof.

13. A method for the treatment of disorders of lipid and carbohydrate metabolism comprising the administration of one or more compounds recited in claim 1 to a patient in need thereof.

14. A method for the treatment of lipid and carbohydrate metabolism disorders comprising the administration of one or more compounds recited in claim 4 to a patient in need thereof.

15. A method for the treatment of arteriosclerosis and the physical manifestations thereof comprising the administration of one or more of the compounds recited in claim 4 to a patient in need thereof.

16. A method for the treatment of arteriosclerosis and the physical manifestations thereof comprising the administration of one or more of the compounds recited in claim 4 to a patient in need thereof.

17. A method for the treatment of insulin resistance comprising the administration of one or more of the compounds recited in claim 1 to a patient in need thereof.

18. A method for the treatment of insulin resistance comprising the administration of one or more of the compounds recited in claim 4 to a patient in need thereof.

19. A method for the treatment of abnormal blood glucose levels comprising the administration of one or more of the compounds recited in claim 8 to a patient in need thereof.

20. A method for the treatment of type II diabetes comprising the administration of one or more of the compounds recited in claim 8 to a patient in need thereof.

21. A method for the treatment of lipid and carbohydrate metabolism disorders comprising the administration of one or more of the compounds recited in claim 8 to a patient in need thereof.

22. A method for the treatment of abnormal blood glucose levels comprising the administration of one or more of the compounds recited in claim 4 to a patient in need thereof.

23. A process for the manufacture of a pharmaceutical composition comprising one or more of the compounds recited in claim 1 comprising the mixing the active ingredient with a pharmaceutically suitable carrier and converting this mixture into a form suitable for administration.
